(19) United States
(12) Patent Application Publication
(10) Pub. No.: US 2019/0350820 A1
(43) Pub. Date: Nov. 21, 2019

(54) Personal Care Compositions

(71) Applicant: Tate & Lyle Ingredients Americas LLC, Hoffman Estates, IL (US)

(72) Inventors: Fengqui Fan, Northbrook, IL (US); Jeremy John Iwanski, Decatur, IL (US); Mark Chandler, Newark, DE (US)

(21) Appl. No.: 16/413,497

(22) Filed: May 15, 2019

Related U.S. Application Data
(60) Provisional application No. 62/671,899, filed on May 15, 2018.

(51) Int. Cl. A61K 8/06; A61K 8/73; A61K 8/34

(52) U.S. Cl. CPC A61K 8/06; A61Q 5/02; A61K 8/345; A61K 8/732

(57) ABSTRACT

Described is a personal care composition comprising: a propanediol or an ester thereof; and a modified starch selected from the group consisting of modified corn starch such as modified dent corn starch or modified waxy corn starch; modified wheat starch; modified barley starch; modified tapioca starch; modified triticale starch; modified sorghum starch; modified rice starch; and modified legume starch such as modified pea starch or modified soybean starch. Uses and methods of using such modified starches in personal care compositions are also described. Advantageously, the propanediol and/or the modified starch can be bio-based, and thus can provide for formulations including even higher contents of materials based on natural sources such as plant sources.

### CROSS-REFERENCE TO RELATED APPLICATIONS

This application claims the benefit of priority of U.S. Provisional Patent Application 62/671,899, filed May 15, 2018, which is hereby incorporated herein by reference in its entirety.

### FIELD OF THE INVENTION

The present invention relates to personal care compositions, particularly personal care compositions containing modified starch and/or propane diol or esters thereof, especially modified starches and/or propanediols that are based on natural sources and have a high degree of bio-based carbon.

### BACKGROUND

Personal care compositions are generally applied to surfaces of the human body, and are particularly applied to the skin or to the hair. According to their use, personal care compositions are tailored to have desirable sensory properties. In particular, personal care compositions are desired to have one or more of specific tactile properties, scent properties and visual properties. For example, soap for personal care is often desired to be smooth, soft to the touch, and perfumed. Furthermore, personal care compositions are tailored to have desirable physical properties, such as specific rheological properties, gelling properties, emulsion properties, foaming properties, viscosity, etc. Naturally, the properties desired for the personal care composition will depend on the use of said personal care composition.

However, personal care compositions known in the art often contain relatively harsh chemicals (e.g. surfactants) which may damage the skin or may be harmful to the environment. Some personal care compositions containing such chemicals may leave the area to which the composition is applied feeling dry, inflamed, irritated or sore. Said harsh chemicals may include, for example, sulfates, sulphites, nitrates, nitrites, thiosulfates, plastics and the like. Plastics may include synthetic polymers such as, but not limited to, polyvinylchloride, polyethylene, polypropylene, polystyrene, and the like.

Personal care compositions are washed into waste water treatment systems when the user of the personal care composition showers or bathes. Ultimately, the plastics and chemicals present in the personal care compositions can pollute the waterways, can infiltrate food chains by ingestion, and can disrupt ecosystems. It is especially desirable to eliminate the need for plastic microbeads as these can pollute waterways for long periods of time and accumulate in tissue if ingested.

As such, there is a desire for improved personal care compositions which are more biodegradable and/or which replace harsh chemicals or plastics with naturally occurring, naturally sourced and/or biodegradable materials.

US 2015/132247 describes cosmetic care compositions comprising a polymer selected from polyvinyl alcohols, vinylpyrrolidone polymers and copolymers and latex, along with at least one starch and at least one polyol. U.S. Pat. No. 8,309,116 describes personal care compositions comprising esters of naturally occurring 1,3-propanediol. U.S. Pat. No. 8,048,920 and US 2015/125403 describe personal care compositions comprising naturally occurring 1,3-propanediol or esters thereof.

There is therefore a desire to provide a component for new personal care compositions which address one or more of the aforementioned problems. It is desirable for instance to provide new personal care compositions that exhibit improved tactile properties. In particular, there is a desire to replace conventional materials in personal care compositions, such as synthetic polymers and surfactants, with components for personal care compositions which are biodegradable, naturally-occurring/naturally-derived, readily available, and ideally without adversely affecting the desired physical properties of the composition.

It is an object of the present invention to obviate or mitigate one or more of the disadvantages associated with personal care compositions described above.

### SUMMARY OF INVENTION

At its most general the invention proposes the use of certain modified starches and optionally propanediol or esters thereof for improving the properties of personal care compositions. Particularly advantageous results are observed when modified starches and propanediol (especially 1,3-propanediol) or esters thereof are used in combination. The inventors have found that these components are useful for imparting desirable properties to respective carriers, making them eminently suitable for use in personal care compositions, e.g. for topical administration. The inventors have also found that combinations of modified starch and propanediol or esters thereof can be used to substantially, or entirely, replace certain undesirable agents conventionally used in personal care compositions, e.g. synthetic polymers or surfactants such as sulfates, whilst retaining desirable composition properties, e.g. tactile properties. This makes these materials useful in replacing sulphate compounds in personal care compositions, such as commonly used in shampoos. As starch and propanediol (e.g. propan-1,3-diol) can be obtained from natural sources and are biodegradable, this makes the resulting compositions more environmentally friendly than prior art compositions without forfeiting tactile performance, and often without imparting any additional odour to the composition.

Various modified starches as described herein have been found to be particularly useful as rheology modifiers, thickeners, gelling agents, emulsion stabilisers, foam stabilisers and/or film formers in various personal care product applications. Particularly desirable results have been observed when such modified starches are used in combination with propanediol, particularly 1,3-propanediol.

Advantageously, modified starch and 1,3-propanediol do not chemically react with each other or with other common chemical components of carriers under mild formulation conditions for personal care products.

The use of modified starches as described herein and/or propanediol or esters thereof have been found by the present inventors to impart pleasing tactile properties that are similar to existing synthetic, petroleum-derived additives, without adding disagreeable color or odor or other detrimental side effects. More specifically, particular combinations of modified starches, and combinations that include propanediol (and/or related esters) especially 1,3-propanediol, have been found to impart desired feel and performance within a broad class of personal care products (skin and hair care, primarily). Improved product attributes include amongst others: improved viscosity without stringiness or globular domains, smooth spreading, more uniform texture, non-greasy feel, improved emulsion stability, compatibiliser for other ingredients, improved fragrance, foam stability, better adhesion to skin and hair, wear resistance, etc. Other advantages will be apparent from the present disclosure.

Exemplary personal care compositions include emulsions, lotions, creams (including sun-blocking products), serums, cleansers, hair products (e.g. styling gels and shampoos), and cosmetics. Other suitable personal care compositions are described herein.

The present application describes a personal care composition comprising:
a propanediol or an ester thereof; and
a modified starch selected from the group consisting of modified corn starch such as modified dent corn starch or modified waxy corn starch; modified wheat starch; modified barley starch; modified tapioca starch; modified triticale starch; modified sorghum starch; modified rice starch; and modified legume starch such as modified pea starch or modified soybean starch.

Also described is the use of a modified starch as a rheology modifier, thickener, gelling agent, film forming agent, emulsion stabilising agent, or foam stabilising agent in a personal care composition, wherein the modified starch is selected from one or more of the following: a modified corn starch such as modified dent corn starch or modified waxy corn starch; modified wheat starch; modified barley starch; modified tapioca starch; modified triticale starch; modified sorghum starch; modified rice starch; and modified legume starch such as modified pea starch or modified soybean starch. Optionally the composition further comprises propanediol or an ester thereof.

Further described is a method of modifying the rheological properties, thickness, gelling properties, film forming properties, emulsion stability or foam stability of a carrier in a personal care composition, the method comprising adding to the carrier
a modified starch and optionally a propanediol or an ester thereof,
to form a personal care composition having respectively modified rheological properties, thickness, gelling properties, film forming properties, emulsion stability or foam stability relative to the composition prior to addition of the modified starch and optionally a propanediol or an ester thereof to the carrier, wherein the modified starch is selected from one or more of the following:
a modified corn starch such as modified dent corn starch or modified waxy corn starch; modified wheat starch; modified barley starch; modified tapioca starch; modified triticale starch; modified sorghum starch; modified rice starch; and modified legume starch such as modified pea starch or modified soybean starch.

Modified starches for use in the present compositions, uses and methods of the invention are described in more detail herein.

Preferred modified starches for use in personal care compositions (e.g. the compositions, uses and methods of the invention disclosed herein) include:
a) waxy corn starch that is hydroxypropylated and cross-linked (e.g. by phosphorylation); and
b) dent corn starch that is hydroxyethylated; and
c) maltodextrin; and
d) waxy corn starch that is cross-linked (e.g. by phosphorylation).

These modified starches in particular have been found to demonstrate surprisingly advantageous tactile properties in personal care compositions, especially when used in combination with propanediol or esters thereof, and especially so when used in combination with 1,3-propanediol, e.g. Bio-1,3-propanediol.

The modified starch may for instance be a). Advantageously it has been found by the inventors that modified starch of the type “a” is useful as a rheological modifier or thickener for personal care compositions according to the invention. Such modified starch provides a smooth emulsion viscosity when used in emulsions. It has surprisingly been found to be able to replace Xanthan gum or carbomer in comparative care compositions without detriment to the tactile properties of the composition. When modified starch “a” is used, the composition may thus in some embodiments be absent of Xanthan gum and/or carbomer. This modified starch has also been found to be able to provide viscosity to the composition suitable for providing a “serum” (i.e. a fluid having lower viscosity than a gel and can be used from a tube but doesn't run when applied to the skin).

The modified starch may be b). Advantageously it has been found by the inventors that modified starch of the type “b” is particularly useful as a film former in compositions of the invention, or as a thickener when used in higher concentrations (see examples). Modified starch of the type “b” has particularly been found to be useful as a fixative (e.g. in hair set compositions such as hair gels). It can provide emulsion stability and foam stability to compositions (see examples). As such, this modified starch has been found to be able to replace sulfate surfactants in personal care compositions. When modified starch “b” is used, the personal care compositions may thus comprise an absence of sulfate compounds. Starch “b” has been found to be particularly advantageous when used in combination with starch “a”.

The modified starch may be c). Advantageously it has been found by the inventors that modified starch of the type “c” is particularly useful as a film former in compositions of the invention. It has been found to be particularly useful in increasing wear of compositions of the invention, and as a suspending agent (see examples). This starch has been found to reduce stickiness of compositions and so provides particularly desirable results when included in sunscreens and make up compositions. Examples of compositions that are deemed to particularly suitable for starch “c” based on these properties include: sun screens, moisturizing lotions, acne creams, mascara, lipsticks, foundations, various ointments, antiperspirants, etc.

The modified starch may be d). Advantageously it has been found by the inventors that modified starch of the type “d” is useful as thickener or gel former for personal care compositions according to the invention (see examples). Such modified starch has been found to be useful in providing clear gels and serums, and so is eminently suitable for use in eye care products, e.g. eye gels. Personal care compositions containing such modified starch and optionally propanediol or ester thereof may therefore in embodiments be a gel.

These starches have been found to impart particularly advantageous properties when used in combination. Thus, also described is a personal care composition comprising at least two modified starches selected from the group consisting of
a) a waxy corn starch that is hydroxypropylated and cross-linked (e.g. by phosphorylation); and
b) a dent corn starch that is hydroxyethylated; and
c) a maltodextrin; and
d) a waxy corn starch that is cross-linked (e.g. by phosphorylation),
optionally wherein the composition further comprises propanediol or an ester thereof. The modified starch may be provided as a combination of modified starches a+b, a+c, a+d, b+c, b+d, c+d, a+b+c, a+b+d, b+c+d. A particularly preferred combination of said modified starches is a+b. This combination of modified starches in particular has been found to demonstrate advantageous and cooperative properties when used in compositions, and especially when used with 1,3-propanediol, as described further in the examples section.

Notably, the propanediols and modified starches described herein can be based on natural sources, and thus can contain a high degree of bio-based carbon. Thus use of such bio-derived propanediols and modified starches can provide for formulations based even more substantially on natural sources such as plant sources.

Any description or limitation herein of the modified starch component, propanediol or ester thereof, or description of the composition in general may be applied analogously to any of the other aspects of the invention, i.e. to the above compositions per se, or to the compositions as described in relation to the disclosed uses and methods.

These aspects and further embodiments of the invention are described in further detail below.

### DETAILED DESCRIPTION OF THE INVENTION

In an aspect of the invention is provided a personal care composition comprising:
a propanediol or an ester thereof; and
a modified starch selected from the group consisting of modified corn starch such as modified dent corn starch or modified waxy corn starch; modified wheat starch; modified barley starch; modified tapioca starch; modified triticale starch; modified sorghum starch; modified rice starch; and modified legume starch such as modified pea starch or modified soybean starch.

### Propanediol or Esters Thereof

Preferably the personal care compositions described herein (either per se or in relation to the uses and methods discussed) comprise a propanediol. In some embodiments, the composition may be absent propanediol esters.

Propanediols are available from a number of manufacturers and may be produced via known synthetic methods. The propanediol is typically 1,3-propanediol. Propanediols, particularly 1,3-propanediol, when combined with the modified starches described herein, have been found by the inventors to result in personal care compositions that are described by subjects as “feeling better” on the skin compared to compositions formed using glycerine or other polyols (e.g. other glycols such as propylene glycol) instead of propanediol.

Moreover, propanediol, particularly 1,3-propanediol, has been found to ad as a surprisingly good solubilising agent for the modified starches of the invention, particularly in the case of hydroxypropylated crosslinked (e.g., phosphorylated) waxy corn starch. This is advantageous as it allows the starch to be added more quickly to the compositions at the point of formulation, thus increasing formulation efficiency, which may be significant on an industrial scale.

The 1,3-propanediol may be obtained from biological sources. Preferably the 1,3-propanediol is biologically produced or biologically derived 1,3-propanediol, i.e. “bio-PDO”), optionally derived by fermentation methods. Fermentation may for instance be by the metabolism of plant derived sugars. Such sugars are typically composed of carbon of atmospheric origin. Bio-1,3-propanediol and its esters may for instance be provided according to the methods described in U.S. Pat. No. 8,048,920B2 or U.S. Pat. No. 8,309,116B2, both of which are incorporated herein by reference. Such biologically sourced 1,3-propanediol is referred to herein interchangeably as “bio-1,3-propanediol”, “Bio-PDO” and “BPDO”. Bio-1,3-propanediol can thus represent a useful plant-derived substance, to provide for formulations based even more substantially on natural sources such as plant sources.

It is especially preferred that 1,3-propanediol of the present invention is BPDO. BPDO is produced using more environmentally benign biological process compared to the methods used to manufacture synthetic glycols and avoids trace contamination with impurities associated with petroleum products, which are often used as starting materials in synthetic preparation of glycols. BPDO is typically produced in a liquid form and offers dispersive properties similar to propylene glycol when added with starch during mixing. The combination of BPDO and modified starches according to embodiments of the invention provides for a “greener” family of personal care products. A particularly suitable source of BPDO is provided under the trade name Zemea® by DuPont Tate & Lyle.

Biologically derived 1,3-propanediol contains biobased carbon. All three carbon atoms in bio-1,3-propanediol will thus be biobased carbons. If a conjugate ester is formed using carboxylic acids that contain all biobased carbon, then the resulting esters also contain all biobased carbon. If, however, the carboxylic acids contain non-biobased carbons, i.e. carbons from a fossil fuel source, then the resulting ester will contain a percentage of biobased carbon in proportion to the number of carbons contributed from the carboxylic acid compared to the three carbons contributed from the biologically-derived 1,3-propanediol. Assessment of the biobased carbon in a material can be performed through standard test methods. Using radiocarbon and isotope ratio mass spectrometry analysis, the biobased content of materials can be determined. ASTM International, formally known as the American Society for Testing and Materials, has established a standard method for assessing the biobased content of materials. The ASTM method is designated ASTM-D6866. The application of ASTM-D6866 to derive a “biobased content” is built on the same concepts as radiocarbon dating, but without use of the age equations. The analysis is performed by deriving a ratio of the amount of radiocarbon (14C) in an unknown sample to that of a modern reference standard. The ratio is reported as a percentage with the units “pMC” (percent modern carbon). If the material being analyzed is a mixture of present day radiocarbon and fossil carbon (containing no radiocarbon), then the pMC value obtained correlates directly to the amount of Biomass material present in the sample. Esters containing a higher degree of biobased carbon are preferred for use in the present invention.

Suitable esters of the propanediol include any conjugate mono or diester that is suitable for use in personal care compositions. The esters may be formed by reaction of propanediol with a respective acid counterpart using chemistry that is well known to the skilled person (e.g. under acid catalysed conditions or dehydrating conditions). Appropriate carboxylic acids for producing esters of 1,3-propanediol may generally include: (1) C1-C3 carbon-containing monocarboxylic acids, including formic acid and acetic acid; (2) fatty acids, such as those acids containing four or more carbon atoms; (3) saturated fatty acids, such as butyric acid, caproic acid, valeric acid, caprylic acid, capric acid, lauric acid, myristic acid, palmitic acid, stearic acid, arachidic acid, and behenic acid; (4) unsaturated fatty acids, such as oleic acid, linoleic acid, and euricic acid; (5) polyunsaturated fatty acids, such as alpha-linolenic acid, stearidonic acid (or moroctic acid), eicosatetraenoic acid, omega-6 fatty acids, arachidonic acids, and omega-3 fatty acids, eicosapentaenoic acid (or timnodonic acid), dosocapentaenoic acid (or clupanodonic acid), and docosahexaenoic acid (or cervonic acid); (6) hydroxy fatty acids, such as 2-hydroxy linoleic acid, and recinoleic acid; phenylalkanoic fatty acids, such as 11-phenyl undecanoic acid, 13-phenyl tridecanoid acid, and 15-phenyl tridecanoid acid; and (7) cyclohexyl fatty acids, such as 11-cyclohexyl undecanoic acid, and 13-cycohexyl tridecanoic acid.

The following acids and their salts or alkyl esters are particularly useful: acetic, alginic, butyric, lauric, myristic, palmitic, stearic, arachidic, adipic, benzoic, caprylic, maleic, palmitic, sebacic, archidonic, erucic, palmitoleic, pentadecanoic, heptadecanoic, nondecanoic, octadectetraenoic, eicosatetraenoic, eicosapentaenoic, docasapentaenoic, tetracosapentaenoic, tetrahexaenoic, docosahexenoic, (alpha)-linolenic, docosahexaenoic, eicosapentaenoic, linoleic, arachidonic, oleic, erucic, formic, propionic, valeric, caproic, capric, malonic, succinic, glutaric, adipic, pimelic, suberic, azelaic, tartaric, citric, salicylic, acetylsalicylic, pelargonic, behenic, cerotic, margaric, montanic, melissic, lacceroic, ceromelissic, geddic, ceroplastic undecylenic, ricinoleic, and elaeostearic acid as well as mixtures of such acids. A more preferred list of suitable organic acids are acetic, adipic, benzoic, maleic, sebacic, and mixtures of such acids. A more preferred list of suitable “fatty acids” meaning generally acids named containing 8-40 carbon in the carbon useful in the present invention include butyric, valeric, caproic, caprylic, pelargonic, capric, lauric, myristic, palmitic, stearic, arachidic, behenic, cerotic, oleic, linoleic, linolenic, margaric, montanic, melissic, lacceroic, ceromelissic, geddic, ceroplastic and the mixtures of such acids. Among those acids, these acids, and their salts and alkyl esters are most preferred stearic, lauric, palmetic, oleic, 2-ethyl hexanoic, and 12-hydroxystearic and mixtures of such acids.

The ester may for instance be selected from optionally substituted C₁₋₁₀alkyl esters, C₁₋₁₀alkenyl esters, and C₁₋₁₀alkylether esters. Exemplary esters include distearate and monostearate, dilaurate and monolaurate, dioleate and monooleate, divalerate and monovalerate, dicaprylate and monocaprylate, dimyristate and monomyristate, dipalmitate and monopalmitate, dibehenate and monobehenate, adipate, maleate, dibenzoate, diacetate and the like. Where the ester is a diester, the ester may be a mixed ester (i.e. wherein a different conjugate ester group is on each hydroxyl group of the diol), but typically the ester groups of a diester are the same.

The personal care composition as described herein (with reference to the compositions per se or the use or methods which describe a personal care composition) may comprise up to about 80% wt of propanediol or ester thereof by dry mass relative to the dry mass weight of the personal care composition, optionally from about 10-65%, such as from about 20 to 65% wt by dry mass. In embodiments, the propanediol or ester thereof may be present in an amount of from 30 to 40% wt by dry mass such as around 35% or 37% wt. by dry mass. Alternatively, the propanediol or ester thereof may be present in an amount of from 20 to 30% wt by dry mass, such as around 25% wt by dry mass. Alternatively, the propanediol or ester thereof may be present in an amount of from 50 to 70% wt by dry mass, such as around 55 to 65% wt by dry mass, e.g. around 60% wt or 64% wt by dry mass.

In embodiments, the propanediol or ester thereof may be provided in an amount of up to about 50% wt relative to the total weight of the personal care composition, optionally from about 5-30%, such as from about 10-25%, or 13-18% wt, and preferably about 15% wt relative to the total weight of the personal care composition.

The following passage provides some exemplary amounts of propanediol or ester thereof, e.g. 1,3-propanediol, e.g. BDPO for certain end use formulations. These are exemplary and non-limiting:

Baby products, such as, for example, baby shampoos, soaps, wipes, lotions, oils, powders, and creams, or bath products such as for example, bath oils, tablets, and salts; bubble baths and bath capsules, may in examples contain propanediol concentration ranges from about 0.1% to about 25% by weight, such as from about 1% to about 10% by weight, e.g. 1 to 5%.

Eye makeup preparations such as, for example, eyebrow pencil; eyeliner; eye shadow; eye lotion; eye makeup remover; and mascara, may in examples comprise from about 0.001% to about 75%, such as 0.01% to about 25%, e.g. 0.05% to about 5% wt propanediol or ester thereof. Makeup preparations (not eye), for example, blushers (all types); foundations; leg and body paints; lipstick; makeup bases; rouges; and makeup fixatives, may in examples contain propanediol or an ester thereof in a concentration range of from about 0.001% to about 99%, such as from about 0.01% to about 25%, e.g. from about 0.05% to about 10%.

### Modified Starches

It will be understood that any one, or a combination of two or more, of the modified starches recited in the aspects of invention above may be used in the compositions, uses and methods of the present invention. The personal care composition may contain only one such modified starch. The personal care composition may comprise more than one of said modified starches. For instance, the composition may comprise 2, 3, 4 or 5 of said modified starches. Where multiple modified starches are present, there are typically 2 or 3, e.g. 2. A particularly useful combination has been found to be: a) a waxy corn starch that is hydroxypropylated and cross-linked (e.g. by phosphorylation); and b) a dent corn starch that is hydroxyethylated, and optionally c) a maltodextrin. Advantages of using such a combination are described further in the examples section.

It will be appreciated that the compositions of the invention (as described in the compositions, uses and methods herein) may include other starches in addition to those modified starches of the invention recited herein, e.g. in the claims. In preferred embodiments, the personal care composition does not contain additional starches in addition to those modified starches of the invention recited herein, e.g. in the claims. The personal care compositions of the invention as described in the compositions, uses and methods herein are typically devoid of natural, i.e. unmodified starches. The personal care composition may be devoid of modified starches other than those modified starches of the invention recited herein, e.g. in the claims.

It is preferred that the modified starch is selected from the group consisting of modified dent corn starch, modified waxy corn starch and modified tapioca starch. It is particularly preferred that the modified starch be modified dent corn starch and/or modified waxy corn starch.

It will be understood that reference to a “modified starch” refers to a base starch that has been modified by chemical and/or physical modification. The term “modified corn starch” for instance describes a corn starch (i.e. a starch obtained from corn) that has been modified as described. Starches of the present invention are typically derived from base starches by chemical modification. Chemically modified starches may additionally be further modified, such as by physical modification.

The base starches may for instance be acidically or enzymatically treated (also known as “acid thinned” or “enzyme thinned” respectively), so as to hydrolyse the starch (e.g. to form maltodextrin). The modified starch in embodiments of the invention may thus be hydrolysed starch, such as enzymatically hydrolysed starch, acid-catalysed hydrolysed starch or any other suitable hydrolysed starch known to those skilled in the art. Examples of hydrolysed starches include maltodextrins.

The starch may alternatively, or additionally, be functionally modified. Such functional modification of starch is known to the skilled person and may include one or more of: alkylation (e.g. propylation or ethylation) to form hydroxyalkylated starches (e.g. “hydroxypropylated”, or “hydroxyethylated” starch), cross-linking and phosphorylation.

Modified starch according to any compositions, use, or method of the invention may selected from the group consisting of hydrolysed starch, oxidized starch, esterified starch, etherified starch, acetylated starch, C₁₋₁₀alkylated starch, hydroxyC₁₋₁₀alkylated starch, crosslinked starch, phosphorylated starch, succinylated starch, and a starch that contains two or more of said modifications. In other words, this list describes suitable exemplary modifications for the modified starches of the present invention. The modified starch may be amphoteric. As an example, where a starch is said to be acetylated, this means that it has been modified so as to include pendant acetyl groups. Succinylated starch by analogy refers to starch modified to include a pendant succinyl moiety. This may be for instance the result of a reaction between starch and a succinyl containing group, e.g. octenyl succinyl anhydride (e.g. the modified starch may be an octenyl succinylated modified starch). A suitable example of such a starch product is “Aq™ Star provided by Aqdot®, Cambridge, UK.

The modified starch may in preferred embodiments be a hydroxyC₁₋₁₀alkylated starch, such as a hydroxyC₂₋₄alkylated starch, and is preferably selected from one or more of hydroxypropylated and hydroxyethylated starch. In a preferred embodiment the composition comprises a hydroxypropylated starch (e.g. a hydroxypropylated corn starch).

Phosphorylation of starch is intended to mean the addition of phosphorous containing moieties such as phosphate, or phosphate esters. Depending on the reagents and methods used, the phosphate moiety may for instance result in addition of a pendant phosphate or phosphate ester group. On the other hand, phosphorylation may be used as a method of crosslinking the starch (e.g. by introduction of cross-linking phosphate groups). Such starches would be both crosslinked and phosphorylated. Cross linking may be induced by any suitable method known to the skilled person. Phosphorylation is a particularly preferred method for cross-linking starches according to the invention. For instance, phosphorous oxychloride may be used to cross link starch. Suitable cross linking groups include phosphate (i.e. to form a phosphorylated starch).

The modified starch may in particularly preferred embodiments be selected from one or more of the following:
a) a waxy corn starch that is hydroxypropylated and cross-linked (e.g. by phosphorylation); and
b) a dent corn starch that is hydroxyethylated; and
c) a maltodextrin; and
d) a waxy corn starch that is cross-linked (e.g. by phosphorylation).

The modified starch may for instance be a). The modified starch may be b). The modified starch may be c). The modified starch may be d). Advantages of these modified starches when used in personal care compositions according to the invention are described under “Summary of invention” and in the “Examples” section. The modified starch may be provided as a combination of modified starches a+b, a+c, a+d, b+c, b+d, c+d, a+b+c, a+b+d, b+c+d. A preferred combination of said modified starches is a+b. Another desirable combination is a combination of modified starches a+b+c.

As described above, modified starches of the invention, such as the chemically modified starches described may be physically modified. Physical modification includes drying (e.g. by spray drying, roll-drying or drum drying), annealing, agglomerating, recrystallizing, and extruding, etc. These modification methods are well known to persons having ordinary skill in the art. Typically, the modified starches of the invention may be both chemically and physically modified.

Advantageously, the starches of the disclosure can be biologically-derived, and thus can contain a high degree of biobased carbon. As described in detail below, the base starches can be obtained from natural sources such as corn. Even when a modification (e.g., hydroxyethylation, hydroxypropylation) is performed using non-bio-based materials, the overall proportion of bio-derived carbon can be very high because the base starch makes up a high percentage of the overall material. Starches containing a higher degree of biobased carbon are preferred for use in the present invention. Thus, bio-based starches like those described herein can represent useful plant-derived substances, to provide for formulations based even more substantially on natural sources.

Modified starches for use in the present invention may be obtained by extracting the base starch from the relevant natural source using standard wet milling processes (which are well-known to persons with ordinary skill in the art), followed by suitable modification. Exemplary natural sources include but are not limited to: sorghum, wheat, pea, corn (dent and waxy), and tapioca, with corn starch (a.k.a. Maize starch) and tapioca starch being particularly preferred base starches.

The modified starch may be sourced from genetically modified or non-genetically modified sources. The starch source may for instance be non-genetically modified starch, e.g. the modified starch may be obtained from a non-genetically modified source of corn starch.

The modified starches for use according to the invention may be supplied as liquid solutions or water soluble powders. They may be added to personal care formulations at various different addition points during formulation, lending flexibility to the manufacturing process.

The modified starches may be added to the composition with discernible effect in the range of about 0.1 to about 20 percent by weight relative to the weight of the composition. Higher amounts of starches may be provided for gel compositions, such as up to 99% by weight. Modified starches of the invention may in embodiments be used as a thickener as they have been found to dissolve easily and build viscosity of compositions such as creams and lotions.

In embodiments of the personal care compositions as described herein, or the recited uses or methods which describe a personal care composition, the modified starch may be provided in the composition in an amount of up to about 99% wt of the modified starch by dry mass relative to the total dry mass weight of the personal care composition, optionally from up to about 80%, such as up to about 70%, up to about 60% or up to about 50% wt. by dry mass. Optionally the modified starch is provided in an amount of from about 1-30%, such as from about 1-15%, and in embodiments, from about 1-10% wt by dry mass relative to the total dry mass weight of the personal care composition. The modified starch may for instance be present in an amount of around 4 to 7% wt by dry mass relative to the total dry mass weight of the composition. In other embodiments, the modified starch may be present in an amount of from 10% to 30% wt by dry mass such as 12 to 28%. For certain compositions such as gels, it may be preferable to include a higher amount of starch, such as 20% to 99% wt by dry mass relative to the dry mass weight of the composition. The modified starches may be added to the composition in an amount of from about 10 to 35% weight by dry mass relative to the dry mass weight of the composition, such as around 18 to 30% wt. In an exemplary embodiment, the composition contains from 20% to 30% wt by dry mass of the modified starch (e.g. approx. 26%) relative to the dry mass weight of the composition. In another exemplary embodiment, the composition contains from 18% to 22% wt modified starch by dry mass weight (e.g. approx. 21%) relative to the dry mass weight of the composition. In another exemplary embodiment, the composition contains from 3 to 8% wt modified starch by dry mass weight (e.g. approx. 5% wt) relative to the dry mass weight of the composition. In another exemplary embodiment, the composition contains from 18-22% wt of the modified starch by dry mass (e.g. approx. 20% wt) relative to the dry mass weight of the composition.

In embodiments the modified starch may be provided in an amount of up to about 90% wt, 80% wt, 70% wt, 60% wt, or 50% wt relative to the total weight of the personal care composition, optionally from about 1-25%, such as from about 1-15%, and in embodiments from about 1-10%. The modified starch may for instance be present in an amount of around 2 to 10, such as around 3 to 7% wt relative to the total weight of the composition. The modified starches may be added to the composition in an amount of about 1 to about 40 weight percent relative to the weight of the composition, but are typically added in an amount of no more than around 10% relative to the weight of the composition. In an exemplary embodiment, the composition contains from 3-8% wt starch (e.g. approx. 5 or 6.5% wt) relative to the weight of the composition. In another exemplary embodiment, the composition contains from 2-6% wt starch (e.g. approx. 3% wt) relative to the weight of the composition. In another exemplary embodiment, the composition contains from 6-10% wt starch (e.g. approx. 8% wt) relative to the weight of the composition.

The weight ratio of propanediol or ester thereof to modified starch in the compositions of the invention per se or the compositions as described in relation to the uses and methods of the invention, may be from 1:10 to 10:1, optionally from 1:5 to 8:1, such as from about 1:1 to 6:1 (e.g. around 5:1), for instance from 2:1 to 4:1, e.g. around 3:1, such as about 2.5:1. In embodiments the ratio is around 2:1 to 3:1. In embodiments the ratio is around 4:1 to 6:1. In embodiments the ratio is around 1:1 to 2:1. Preferably, the propanediol or ester thereof is provided in a % wt amount that is greater than the amount of starch in the composition. Preferably, the propanediol or ester thereof is present in an amount of at least 1.5×, or 2×, or 3× more than the corresponding amount of starch in the composition.

The modified starch may be a thickening starch wherein the personal care composition does not further comprise an additional thickening agent. The modified starch may be a gelling starch wherein the personal care composition does not further comprise an additional gelling agent.

### Personal Care Compositions

The personal care composition may be a fluid (e.g. liquid) composition. The fluid composition may be selected from the group consisting of a gel, a cream, a solution, a spray, a lotion, an ointment, an emulsion, a mousse, a cleanser, a slurry or a paste.

It will be appreciated that personal care compositions as described herein will typically comprise a carrier, preferably a fluid carrier, into which the components of the composition are dispersed and/or dissolved. The term “carrier” is described in further detail below. The carrier typically serves as the medium by which any particular active agents may be delivered, or may itself provide the desirable properties to a subject, such as moisturising properties. In some embodiments, the modified corn starch and propanediol are dispersed or dissolved in the composition in a carrier medium, preferably dissolved.

The personal care composition may be a cosmetic composition, a dermatological care composition, or a body care composition. “Cosmetic compositions” include but are not limited to make-up, lipstick, lip gloss, lip pencil, eye shadow, gel eye colour, eye liner, eye pencil, mascara, concealer, foundation, facial powder, liquid rogues, nail polish, gel polish removers, blush, sunless tanner and bronzing sticks. “Dermatological care compositions” include but are not limited to skin care products, skin cleansing products, facial lotion, moisturiser, body wash, body lotion, foot care, hand cream, cuticle remover, cuticle cream, acne cream, acne cleansing scrub, and sun care formulations like sun screen and sun stick. “Body care compositions” may typically include but are not limited to shampoo, hair conditioner, hair treatment, hair reconstruction, styling gel, styling foam, hair mousse, hair spray, hair set lotion, blow-styling lotion, hair colour lotion and dyes, hair bleaching cream, shaving cream, shaving lotion, hair relaxing, curl activator, fragrant hair gloss, deodorant, antiperspirant, toothpaste, depilatory formulation, facial mask, anti-aging formulation, soap, hand sanitizer, antibacterial hand cleaner, body scrub, hand scrub, bubble bath, bath oils, baby lotion, diaper rash cream, wet wipe, baby bath and vitamin cream.

Exemplary embodiments of personal care compositions include the following compositions “i” to “vii”:

i) A personal care emulsion, wherein;
the propanediol is 1,3-propanediol; and
the modified starch is crosslinked (e.g., phosphorylated) and hydroxypropylated and is optionally a modified waxy corn starch;
optionally wherein the composition does not contain carbomer and/or Xanthan Gum.
   ii) A personal care cleanser, optionally a foaming cleanser, wherein:
the propanediol is 1,3-propanediol; and
the modified starch is crosslinked (e.g., phosphorylated) and hydroxypropylated, preferably wherein the modified starch is modified waxy corn starch.
   iii) A personal care gel or serum, wherein:
the propanediol is 1,3-propanediol; and
the modified starch is crosslinked (e.g., phosphorylated) and hydroxypropylated, preferably wherein the modified starch is modified waxy corn starch.
   iv) A personal care cream, preferably an eye cream, wherein:
the propanediol is 1,3-propanediol; and
the modified starch is crosslinked (e.g., phosphorylated), preferably wherein the modified starch is modified waxy corn starch.
   v) A personal care emulsion, wherein:
the propanediol is 1,3-propanediol; and
the modified starch is hydroxyethylated, preferably wherein the modified starch is modified dent corn starch.
   vi) A sunscreen or makeup, wherein:
the propanediol is 1,3-propanediol; and
the modified starch is maltodextrin.
   vii) A film forming personal care composition wherein:
the personal care composition is a film forming composition;
the propanediol is 1,3-propanediol; and
the modified starch is maltodextrin.

The personal care composition may be lotion comprising:
from about 10-20% by weight bio-1,3-propanediol (e.g. about 15% wt); and
from about 2 to 5% by weight of a modified corn starch selected from one or more of hydroxypropylated and crosslinked (e.g., phosphorylated) waxy corn starch, and hydroxyethylated dent corn starch; and
optionally one or more of the group consisting of emulsifiers, emollients, preservatives, solvents and thickeners. The lotion may for instance be an aqueous lotion, the lotion comprising:
from about 10-20% by weight bio-1,3-propanediol (e.g. about 15% wt); and
from about 1 to 3% by weight of a hydroxypropylated and crosslinked (e.g., phosphorylated) waxy corn starch; and
from about 0.5 to 2% by weight of a hydroxyethylated dent corn starch; and
optionally one or more of the group consisting of emulsifiers, emollients, preservatives, solvents and thickeners. The lotion may in a preferred embodiment be an aqueous lotion comprising:
from about 10-20% by weight bio-1,3-propanediol (e.g. about 15% wt); and
from about 1 to 3% by weight of a hydroxypropylated and crosslinked (e.g., phosphorylated) waxy corn starch; and
from about 0.5 to 2% by weight of a hydroxyethylated dent corn starch; and
from about 0.5% to 4% by weight (e.g. about 3% wt) of a sodium starch octenyl succinate; and
from about 0.2 to 1% (e.g. 0.5%) further emulsifier; and
from about 2 to 3% thickener, such as cetyl alcohol; and
from about 12 to 18% emollient such as heptyl undecylenate; and
from about 0.5 to 2% preservative. The weight ratios provided here are based on the total weight of the composition.

The personal care composition may be a gel (e.g. an eye gel) comprising:
from about 10-20% by weight bio-1,3-propanediol (e.g. about 15% wt); and
from about 4 to 8% by weight of a hydroxypropylated and crosslinked (e.g., phosphorylated) waxy corn starch (e.g. about 6.5%); and
optionally an active agent such as retinyl linoleate in an amount of up to 2% wt (e.g. about 0.1%); and
optionally one or more of the group consisting of emulsifiers, preservatives, emollients, solvents and thickeners. The gel may in a preferred embodiment be an aqueous gel comprising:
from about 10-20% by weight bio-1,3-propanediol (e.g. about 15% wt); and
from about 4 to 8% by weight of a hydroxypropylated and crosslinked (e.g., phosphorylated) waxy corn starch (e.g. about 6.5%); and
from about 1 to 3% emulsifier such as polysorbate (e.g. about 2% wt); and
from about 1 to 2% preservative (e.g. 1.5%); and up to about 2% wt (e.g. about 0.1%) of an active agent such as retinyl linoleate.

The weight ratios provided here are based on the total weight of the composition.

The personal care composition may be a serum (e.g. an eye serum) comprising:
from about 10-20% by weight bio-1,3-propanediol (e.g. about 15% wt); and
from about 3 to 8% by weight of a hydroxypropylated and crosslinked (e.g., phosphorylated) waxy corn starch; and
optionally one or more of the group consisting of emulsifiers, emollients, preservatives, solvents and thickeners. The serum may in a preferred embodiment be an aqueous serum comprising:
from about 10-20% by weight bio-1,3-propanediol (e.g. about 15% wt); and
from about 3 to 8% by weight (e.g. about 5% wt) of a hydroxypropylated and crosslinked (e.g., phosphorylated) waxy corn starch; and
from about 1 to 3% wt emulsifier such as polysorbate (e.g. about 2% wt); and
from about 1 to 2% preservative (e.g. 1.5%); and up to about 2% wt (e.g. about 0.1%) of an active agent such as retinyl linoleate.

The weight ratios provided here are based on the total weight of the composition.

The personal care composition may be a lotion (e.g. a face lotion) comprising:
from about 10-20% by weight bio-1,3-propanediol (e.g. about 15% wt); and
from about 1 to 5% by weight (e.g. about 3% wt) of a modified corn starch selected from one or more of hydroxypropylated and crosslinked (e.g., phosphorylated) waxy corn starch, hydroxyethylated dent corn starch, and maltodextrin; and
optionally one or more of the group consisting of emulsifiers, emollients, preservatives, and solvent. The lotion may for instance be an aqueous lotion, the lotion comprising:
from about 10-20% by weight bio-1,3-propanediol (e.g. about 15% wt); and
from about 0.5 to 2% (e.g. about 1% wt) by weight of a hydroxypropylated and crosslinked (e.g., phosphorylated) waxy corn starch; and
from about 0.5 to 2% by weight (e.g. about 1% wt) of a hydroxyethylated dent corn starch; and
from about 0.5 to 2% by weight (e.g. about 1% wt) of a maltodextrin;
optionally one or more of the group consisting of emulsifiers, emollients, preservatives, solvents and thickeners. The lotion may in a preferred embodiment be an aqueous lotion comprising:
from about 10-20% by weight (e.g. about 15% wt) bio-1,3-propanediol; and
from about 0.5 to 2% by weight (e.g. about 1% wt) of a hydroxypropylated and crosslinked (e.g., phosphorylated) waxy corn starch; and
from about 0.5 to 2% by weight (e.g. about 1% wt) of a hydroxyethylated dent corn starch; and
from about 0.5 to 2% by weight (e.g. about 1% wt) of a maltodextrin;
from about 2 to 8% (e.g. 6%) further emulsifier (e.g. glyceryl stearate and one or more of PEG-100 stearate, or sodium stearoyl lactylate); and
from about 12 to 18% (e.g. about 15% wt) emollient such as isostearyl isostearate; and
from about 0.5 to 2% (e.g. about 1.5%) preservative; and
from about 30-50% wt (e.g. about 40% wt) water. The weight ratios provided here are based on the total weight of the composition.

The personal care composition may be a cream (e.g. a hand cream) comprising:
from about 10-20% by weight bio-1,3-propanediol (e.g. about 15% wt); and
from about 5 to 10% by weight (e.g. about 8% wt) of a modified corn starch selected from one or more of hydroxypropylated and crosslinked (e.g., phosphorylated) waxy corn starch, and hydroxyethylated dent corn starch, and octenyl succinate modified corn starch; and
optionally one or more of the group consisting of emulsifiers, emollients, preservatives, solvents and thickeners. The cream may for instance be an aqueous cream, the cream comprising:
from about 10-20% by weight bio-1,3-propanediol (e.g. about 15% wt); and
from about 2 to 6% (e.g. about 4% wt) by weight of a hydroxypropylated and crosslinked (e.g., phosphorylated) waxy corn starch; and
from about 0.5 to 2% by weight (e.g. about 1% wt) of a hydroxyethylated dent corn starch; and
from about 0.5 to 4% by weight (e.g. about 3% wt) of an octenyl succinate modified corn starch; and
optionally one or more of the group consisting of emulsifiers, emollients, preservatives, solvents and thickeners. The cream may in a preferred embodiment be an aqueous cream comprising:
from about 10-20% by weight (e.g. about 15% wt) bio-1,3-propanediol; and
from about 2 to 6% by weight (e.g. about 4% wt) of a hydroxypropylated and crosslinked (e.g., phosphorylated) waxy corn starch; and
from about 0.5 to 2% by weight (e.g. about 1% wt) of a hydroxyethylated dent corn starch; and
from about 0.5% to 4% by weight (e.g. about 3% wt) of a sodium starch octenyl succinate;
from about 0.2 to 1% (e.g. 0.5%) further emulsifier; and
from about 2 to 3% (e.g. 2.5% wt) thickener, such as cetyl alcohol; and
from about 12 to 18% (e.g. about 15% wt) emollient such as acetylated hydrogenated vegetable glyceride; and
from about 0.5 to 2% (e.g. about 1%) preservative; and
from about 45-65% wt (e.g. about 58% wt) water. The weight ratios provided here are based on the total weight of the composition.

The personal care compositions described herein may contain any suitable further ingredients typically used in personal care compositions. The personal care compositions may for instance comprise one or more of the group consisting of: a carrier, a preservative, an active agent (e.g. therapeutic agent), a nutraceutical agent, an abrasive, an adsorbent, a stabilizer, a colourant, a thickener, a gellant, a solidifying agent, a structuring agent, an anti-foaming agent, a binder, a film former, a humectant, an essential oil, a filler, a surfactant, a plasticiser, an elasticiser, a conditioning agent, an emollient, an astringent, an antiperspirant compound, a sunscreen, a UV absorber, a fragrance, and a dispersant.

The compositions of the invention optionally comprise a safe and effective amount of an active, e.g. for regulating a skin condition (including prophylactically and therapeutically regulating the skin condition). Prophylactically regulating skin conditions includes delaying, minimizing, and/or preventing visible and/or tactile discontinuities in the skin. Therapeutically regulating the skin condiments includes ameliorating e.g., diminishing, minimizing, and/or eliminating such discontinuities. Regulating the skin condition also involves improving the skin appearance and/or feel. Also included is regulating the signs of ageing which can involve prophylactically regulating and/or therapeutically regulating one or more of such signs e.g., fine lines, wrinkles, pores etc.

Methods of treatment of bodily conditions (e.g. skin or hair conditions) are thus contemplated by the present invention wherein the methods comprise administering a composition of the invention containing, or in combination with, a therapeutically effective amount of a suitable active agent suitable for preventing or treating the bodily condition. The invention thus also provides personal care compositions as described herein for use in methods of treatment of bodily conditions (e.g. skin or hair conditions) wherein the method comprises administering a composition of the invention containing, or in combination with, a therapeutically effective amount of a suitable active agent suitable for preventing or treating the bodily condition. Use of the compositions of the invention in methods of manufacturing a medicament for use in treating bodily conditions (e.g. skin or hair conditions) are also described wherein the treatment comprises administering to a subject a composition of the invention containing, or in combination with, a therapeutically effective amount of a suitable active agent suitable for preventing or treating the bodily condition. Typically however the use of the compositions of the invention is for non-therapeutic applications, e.g. cosmetic applications. Ingredients that are known to be useful for regulating skin conditions include vitamin B3 compounds, retinoids, and combinations thereof. The compositions of the present invention may for instance comprise from about 0.05% to about 15% wt, such as about 1 to about 5% of the active.

As described herein, it has been advantageously found by the inventors that the use of modified starches of the present invention, and particularly the use of both a modified starch of the invention and propanediol can provide adequate tactile properties so as to mean that certain conventional ingredients can be omitted from the compositions of the invention without detriment to the desired tactile properties. The personal care composition of any embodiment of the invention may thus be a personal care composition that does not further comprise xanthan gum; and/or an acrylate polymer; and/or a carbomer; and/or a hydrophilic surfactant; and/or a polymer selected from the group consisting of polyvinyl alcohol and vinylpyrollidone polymers; and/or a surfactant; and/or an emulsifier. That is, the composition may be absent any one or more of these ingredients, e.g. all.

In a further aspect of the invention is provided use of a modified starch as a rheology modifier, thickener, gelling agent, film forming agent, emulsion stabilising agent, or foam stabilising agent in a personal care composition, wherein the modified starch is selected from one or more of the following: a modified corn starch such as modified dent corn starch or modified waxy corn starch; modified wheat starch; modified barley starch; modified tapioca starch; modified triticale starch; modified sorghum starch; modified rice starch; and modified legume starch such as modified pea starch or modified soybean starch.

In such use, the modified starch may preferably be used in combination with a propanediol or ester thereof. The propanediol or ester thereof may be as described according to any embodiment herein, such as in the section entitled “Propanediol or esters thereof” or as in the claims.

The modified starch may be as described according to any embodiment of the modified starch described herein, such as under the “Modified starches” section above, or as defined in the claims.

The modified starch may for instance be selected from one or more of the following:
a) a waxy corn starch that is hydroxypropylated and cross-linked (e.g. by phosphorylation); and
b) a dent corn starch that is hydroxyethylated; and
c) a maltodextrin; and
d) a waxy corn starch that is cross-linked (e.g. by phosphorylation).

The use may be as a rheology modifier. The use may be as a thickener. The use may be as a gelling agent. The use may be as a film forming agent. The use may be as an emulsion stabilising agent. The use may be as a foam stabilising agent.

The use may for instance be use as a rheology modifier in a personal care composition, wherein the modified starch is a waxy corn starch that is hydroxypropylated and cross-linked (e.g. by phosphorylation). In such embodiments, the personal care composition may suitably be an emulsion or cleanser.

The use may be as a thickener in a personal care composition, wherein the modified starch is selected from one or more of the following:
a) a waxy corn starch that is hydroxypropylated and cross-linked (e.g. by phosphorylation); and
b) a dent corn starch that is hydroxyethylated; and
c) a waxy corn starch that is cross-linked (e.g. by phosphorylation).

The use may be as a gelling agent in a personal care composition, wherein the modified starch is a waxy corn starch that is cross-linked (e.g. by phosphorylation) and optionally hydroxypropylated.

The use may be as a film forming agent in a personal care composition, wherein the modified starch is selected from one or more of the following:
a) a dent corn starch that is hydroxyethylated; and
b) a maltodextrin.

The use may be as an emulsion stabilising agent in a personal care composition, wherein the modified starch is a dent corn starch that is hydroxyethylated.

The use may be as a foam stabilising agent in a personal care composition, optionally a cleansing composition, wherein the modified starch is a waxy corn starch that is hydroxypropylated and cross-linked (e.g. by phosphorylation).

The use may in typically comprise adding the modified starch to a carrier medium. In some embodiments of the uses of the invention, the personal care composition also comprises propanediol or an ester thereof. The use may thus comprise separately or simultaneously adding modified starch and propanediol or an ester thereof to the carrier medium. When added separately, the starch may be added before or after the propanediol or ester thereof, typically after. It has been found that adding the starch to a composition which already contains at least some propanediol or ester thereof allows for better dispersion/dissolution of the starch in the composition and thus advantageously provides decreased formulation times.

The weight % amounts described elsewhere herein for the modified starches and propanediol or ester thereof in embodiments of compositions of the invention also apply here to embodiments of the use of the invention. In the context of the uses described herein, when the % weight amount of component is stated to be relative to the total dry mass weight of the composition, or relative to the total weight of the composition, this refers to the total weight of the composition once the modified ester and, if present, propanediol or ester thereof have been added.

When propanediol is added to the composition, the weight ratio of propanediol or ester thereof to modified starch in the resulting personal care composition may be from 1:10 to 10:1, optionally from 1:5 to 8:1, such as from about 1:1 to 6:1, for instance from 1.5:1 to 6:1, such as about 5:1. For instance the ratio may be from 2:1 to 4:1, e.g. around 3:1, such as about 2.3:1 or 2.5:1. In embodiments the ratio is around 2:1 to 3:1. In embodiments the ratio is around 4:1 to 6:1. In embodiments the ratio is around 1:1 to 2:1. Preferably, the propanediol or ester thereof is provided in a % wt amount that is greater than the amount of starch in the composition. Preferably, the propanediol or ester thereof is present in an amount of at least 1.5×, or 2×, 3×, 4× or 5× more than the corresponding amount of starch in the composition.

The personal care composition may be further described according to any embodiment described herein, such as in the section entitled “Personal care compositions” or as in the claims.

In a further aspect of the invention is provided a method of modifying the rheological properties, thickness, gelling properties, film forming properties, emulsion stability or foam stability of a carrier in a personal care composition, the method comprising adding to the carrier
a modified starch and optionally a propanediol or an ester thereof,
to form a personal care composition having respectively modified rheological properties, thickness, gelling properties, film forming properties, emulsion stability or foam stability properties relative to the composition prior to addition of the modified starch and optionally a propanediol or an ester thereof to the carrier, wherein the modified starch is selected from one or more of the following:
a modified corn starch such as modified dent corn starch or modified waxy corn starch; modified wheat starch; modified barley starch; modified tapioca starch; modified triticale starch; modified sorghum starch; modified rice starch; and modified legume starch such as modified pea starch or modified soybean starch. The method preferably does include adding to the carrier the propanediol or ester thereof.

In this method and its embodiments, the propanediol or ester thereof may be further defined/limited according to any description of the propanediol of esters thereof described herein, e.g. in relation to the relative weight amounts, etc. for instance as described in the section entitled “Propanediol of esters thereof” or as described in the claims.

In this method and its embodiments, the modified starch may be further described according to any description of the modified starch described herein, e.g. in relation to the relative weight amounts, etc. for instance as described in the section entitled “Modified starches” or as described further in the claims.

Other features of the personal care compositions according to this method may be as described herein for any embodiment of the invention such as in the section entitled “Personal care compositions” or as defined in the claims.

In preferred embodiments, the modified starch is selected from one or more of the following:
a) a waxy corn starch that is hydroxypropylated and cross-linked (e.g. by phosphorylation); and
b) a dent corn starch that is hydroxyethylated; and
c) a maltodextrin; and
d) a waxy corn starch that is cross-linked (e.g. by phosphorylation).

The method may be a method of modifying the rheological properties of the carrier. The method may be a method of modifying the thickness of the carrier. The method may be a method of modifying the gelling properties of the carrier. The method may be a method of modifying the film forming properties of the carrier. The method may be a method of modifying the emulsion stability of the carrier. The method may be a method of modifying the foam stability of the carrier. The modification is, in embodiments, an increase in the relative value of the respective property, e.g. an increase in thickness, emulsion stability, foam stability, etc.

The method may be a method of modifying the theological properties of the carrier, wherein the modified starch is a waxy corn starch that is hydroxypropylated and cross-linked (e.g. by phosphorylation). In exemplary embodiments the personal care composition is an emulsion or cleanser.

The method may be a method of modifying the thickness of the carrier, wherein the modified starch is selected from one or more of the following:
a) one or more waxy corn starches that are hydroxypropylated and cross-linked (e.g. by phosphorylation); and
b) a dent corn starch that is hydroxyethylated; and
c) a waxy corn starch that is cross-linked (e.g. by phosphorylation). In some embodiments, the list does not contain c).

The method may be a method of modifying the gelling properties of the carrier, wherein the modified starch is a waxy corn starch that is cross-linked (e.g. by phosphorylation) and optionally hydroxypropylated.

The method may be a method of modifying the film forming properties of the carrier, wherein the modified starch is selected from one or more of the following:
a) a dent corn starch that is hydroxyethylated; and
b) a maltodextrin.

The method may be a method of modifying the emulsion stability of the carrier, wherein the modified starch is a hydroxypropylated and cross linked (e.g. by phosphorylation) waxy corn starch, or a dent corn starch that is hydroxyethylated.

The method may be a method of modifying the foam stability of the carrier, optionally wherein the personal care composition is a cleansing composition, and wherein the modified starch is a waxy corn starch that is hydroxypropylated and cross-linked (e.g. by phosphorylation).

The method may be a method of converting a personal care lotion to a personal care cream, the method comprising adding to the lotion:
a modified starch and optionally a propanediol or an ester thereof,
to form the personal care cream, wherein the modified starch is as defined according to any embodiment of the modified starches described herein, e.g. wherein the starch is modified corn starch such as modified dent corn starch or modified waxy corn starch; modified wheat starch; modified barley starch; modified tapioca starch; modified triticale starch; modified sorghum starch; modified rice starch; and modified legume starch such as modified pea starch or modified soybean starch. The method preferably includes adding to the carrier the propanediol or ester thereof.

In embodiments wherein propanediol or an ester thereof is added to the carrier, the method may comprise separately or simultaneously adding the modified starch and propanediol or ester thereof to the carrier medium. When added separately, the starch may be added before or after the propanediol or ester thereof, typically after. It has been found that adding the starch to a composition which already contains at least some propanediol or ester thereof allows for better dispersion/dissolution of the starch in the composition and thus advantageously provides decreased formulation times

The modified starch and, if present, the propanediol or ester thereof may be dispersed or dissolved in the carrier, preferably dissolved.

In a further aspect of the invention is provided a personal care composition comprising at least two modified starches selected from the group consisting of:
a) a waxy corn starch that is hydroxypropylated and cross-linked (e.g. by phosphorylation); and
b) a dent corn starch that is hydroxyethylated; and
c) a maltodextrin; and
d) a waxy corn starch that is cross-linked (e.g. by phosphorylation), optionally wherein the composition further comprises propanediol or an ester thereof.

The modified starch may be provided as a combination of modified starches a+b, a+c, a+d, b+c, b+d, c+d, a+b+c, a+b+d, b+c+d, or a+b+c+d. A preferred combination of said modified starches is a+b. The personal care composition may thus preferably comprise:
a) a waxy corn starch that is hydroxypropylated and cross-linked (e.g. by phosphorylation); and
b) a dent corn starch that is hydroxyethylated. Optionally such a composition may further include c) a maltodextrin.

In this aspect and its embodiments, the propanediol or ester thereof may be further defined/limited according to any description of the propanediol of esters thereof described herein, e.g. in relation to the relative weight amounts, etc. for instance as described in the section entitled “Propanediol of esters thereof” or as described further in the claims.

In this aspect and its embodiments, the modified starch may be further described according to any description of the modified starch described herein, e.g. in relation to the relative weight amounts, etc. for instance as described in the section entitled “Modified starches” or as described further in the claims.

When propanediol or ester thereof is added to the composition, the weight ratio of propanediol or ester thereof to modified starch in the resulting personal care composition may be from 1:10 to 10:1, optionally from 1:5 to 8:1, such as from about 1:1 to 6:1, for instance from 1.5:1 to 6:1, such as about 5:1. For instance the ratio may be from 2:1 to 4:1, e.g. around 3:1, such as about 2.3:1 or 2.5:1. In embodiments the ratio is around 2:1 to 3:1. In embodiments the ratio is around 4:1 to 6:1. In embodiments the ratio is around 1:1 to 2:1. Preferably, the propanediol or ester thereof is provided in a % wt amount that is greater than the amount of starch in the composition. Preferably, the propanediol or ester thereof is present in an amount of at least 1.5×, or 2×, 3×, 4× or 5× more than the corresponding amount of starch in the composition.

In embodiments, the composition per se, or the composition as described for the use or methods herein is biodegradable. “Biodegradable” or “Biodegradability” refers to the capacity of a compound to be broken down by living organisms. Usually, this is to form simpler, stable compounds such as carbon dioxide and water. There are many published test methods for measuring the biodegradability of organic chemicals such as glycols. One internationally recognized method is ASTM E1720-01, Standard Test Method for Determining Ready, Ultimate Biodegradability of Organic Chemicals in a Sealed Vessel CO2 Production Test. Chemicals that demonstrate 60% biodegradation or better in this test method will biodegrade in most aerobic environments and are classified as ready biodegradable. Propanediol or its esters thereof as described in this document meet these criteria.

The compositions of the present invention are usually formulated to have a pH of 9.5 or below and in general have a pH in the range from about 4.5 to about 9, more preferably from about 5 to about 8.5, e.g. around 7-8. Some compositions, particularly those comprising an additional active such as salicylic acid, require a lower pH in order for the additional active to be fully efficacious. These compositions are usually formulated to have a pH of from about 2.5 to about 5, more preferably from about 2.7 to about 4

Exemplary conditions of manufacture of the compositions of the invention are described in the specific examples. In exemplary embodiments, a method for preparing a personal care composition is provided, comprising adding one or more modified starches as described according to any embodiment of the modified starch described herein, e.g. as in the claims, to a carrier containing propanediol or an ester thereof. Advantageously, the propanediol (or ester) has been found by the inventors to solubilise the starch in the mixture, thus allowing the starch to be added at an increased rate for improved efficiency.

The compositions of the invention may be administered onto the skin or hair of a subject, i.e. by topical administration. The compositions are typically for use on mammals. Administration to humans is particularly preferred. The methods and uses of the invention in embodiments may thus comprise applying the composition to a mammalian subject, such as a human subject.

Other features of the personal care compositions may be as described herein for any embodiment of the invention such as in the section entitled “Personal care compositions” or as defined in the claims.

Further features of the invention are also described below.

### General

References herein to “% weight by dry mass” refer to the weight of the component relative to the total amount of material in the composition excluding any water. Any water of adsorption and water of crystallisation must therefore be excluded from the weight calculation.

The term “film forming agent” or “film former” are used interchangeably herein. The term will be well known to a skilled person in the field of personal care compositions as an agent that promotes the formation of a continuous covering layer over the hair or skin when applied to their surface. Embodiments of modified starches of the invention are able to provide film forming properties when incorporated into personal care compositions. This may then replace conventional film formers such agents include polyvinylpyrrolidone (PVP), acrylates, acrylamides, and copolymers.

The term “carrier” or “carrier medium” as referred to herein will be well understood in the art by the skilled person. The term “carrier” is analogous to the term “vehicle” as used in other industries to refer to the fluid phase of the composition. In general, the term refers to the basic fluid (e.g. cream, liquid solution or emulsion) of the composition into which the various components of the composition may be dispersed or dissolved.

The carrier may be oil-based or water-based, or may be a dispersion or emulsion (water-in-oil, or oil-in-water). The fluid carrier can range in thickness (viscosity) from flowable liquids to pastes. The additives may also be used in dry powders.

The carrier may represent a lesser or greater proportion of the mass of the personal care product. The fluid carrier may be present in a personal care composition in an amount of from about 1% to 99.5% by weight of the composition. At the top end of this range there would essentially be little active ingredients—e.g. in the case of a moisture-trapping lotion). The lower end of the range may be provided for specific individual applications that contain large amount of active ingredients but require low amounts of carrier.

Preservatives may include one or more of chelants, sequestrants, stabilisers, biocidal compounds, antiseptic compounds, antifungal compounds and antimicrobial compounds.

Therapeutic agents may include one or more of drugs, anti-ageing agents, enzymes, proteins, vitamins, desensitising agents or local anaesthetics (e.g. lidocaine or benzocaine).

Stabilizers may include one or more of pH buffers and pH adjusting agents.

Colourants may include one or more of pigments, opacifiers and dyes.

The carrier may in embodiments comprise, without limitation one or more of the following: moisturizing agents, astringents, chelants and other sequestrants, emollients, preservatives, stabilizers, abrasives, adsorbents, thickeners, gellants, solidifying/structuring agents, anti-foaming agents, pH buffers/adjusting agents, binders, film formers, humectants, pigments, opacifiers, essential oils, fragrances, and aromatic compounds.

The skilled person will be familiar with such ingredients for use in personal care compositions. Exemplary carriers, active agents (including antibacterial actives, anti-inflammatory actives, anti-microbial agents, anti-oxidising agents), chelants, preservatives, thickeners, gellants, structurants, sunscreens, emulsifiers, surfactants, fragrances, colourants, emollients, humectants, etc. for use in compositions of the invention are for instance described in US20150125403A1, which is incorporated herein by reference.

### EXAMPLES

The present invention is further defined by the following examples. Whilst indicating particular embodiments of the invention, such examples are provided by way of illustration only and are not intended to be limiting on the general scope of the invention, which is more broadly defined herein, such as in the accompanying claims.

### 1. Exemplary Starches

The following bio-based modified starches were provided and incorporated into an exemplary personal care composition for performance assessment. Resulting compositions were then observed and tested for tactile properties by application to human skin.

TABLE 1

| | | Observed | |
|---|---|---|---|
| | | function when | |
| | | incorporated into a | Attributes when |
| Modified | | composition of | included in personal |
| Starches: | Modification | the invention | care compositions |
| Starch 1: | Hydroxypropyl | Rheology | Provided smooth |
| TEXTURLUX ™ | starch phosphate | modifying, | emulsion viscosity, |
| Rheo | (crosslinked) | thickening, | Cold processable |
| Waxy corn | | Emulsion | No neutralisation |
| starch | | stabilising | needed |
| (water soluble | | | High shear mixing not |
| powder) | | | required |
| | | | Provides clear gels |
| | | | Non-sticky |
| | | | Especially good results |
| | | | when used in |
| | | | combination with BPDO |
| Starch 2: | Hydrolyzed Corn | Film-forming, | Acts as a fixative |
| TEXTURLUX ™ | Starch | thickening at | Useful for hair set |
| Stabil | Hydroxyethyl Ether | higher solids, | compositions |
| Dent corn starch | | emulsion | Cold processable |
| (water soluble | | stabilising | No neutralisation |
| powder) | | (without | needed |
| | | appreciable | High shear mixing not |
| | | impact on | required |
| | | aesthetics | Can replace sulfate |
| | | | compounds |
| Starch 3: | Hydroxypropyl | Rheology | High viscosity but with |
| TEXTURLUX ™ | starch phosphate | modifying, | smooth flow |
| Hold | (crosslinked) | thickening | Adds lubricity to foam, |
| Waxy corn | | | suspending agent, |
| starch | | | benefits sulfate free |
| (water soluble | | | compositions, forms |
| powder) | | | clear gels |
| Starch 4: | Acid/enzyme | Film forming | Water and wear |
| TEXTURLUX ™ | thinned starch - | | resistance, low |
| Resist | Maltodextrin | | stickiness particularly |
| Maltodextrin | | | when used with |
| (water soluble | | | propanediol, suspending |
| powder) | | | agent, especially |
| | | | beneficial properties |
| | | | observed in combination |
| | | | with BPDO |
| Starch 5: | Hydroxypropyl | Thickening, gel | Provides clear gels |
| TEXTURLUX ™ | starch phosphate | forming | No neutralisation |
| Feel | (crosslinked) | | needed |
| Waxy corn | | | High shear mixing not |
| starch | | | required |
| (water soluble | | | |
| powder) | | | |

The TEXTURLUX™ brand starches described here are bio-based materials, having biobased carbon contents in the range of 94-100%. Accordingly, they represent advantageous plant-derived materials, and can provide for formulations having an even higher degree of bio-based material.

### Performance Summary

Advantageously it has been found by the inventors that starch 1 is useful as a rheological modifier or thickener for personal care compositions according to the invention. Such modified starch provides a smooth emulsion viscosity when used in emulsions. It has surprisingly been found to be able to replace Xanthan gum or carbomer in comparative care compositions without detriment to the tactile properties of the composition. When modified starch “1” is used, the composition may thus in some embodiments be absent Xanthan gum and/or carbomer. This modified starch has also been found to be able to provide viscosity to the composition suitable for providing a “serum” (i.e. a fluid having lower viscosity than a gel and can be used from a tube but doesn't run when applied to the skin). A primary benefit is that it can be added at any time in the process of making an emulsion, and at any suitable temperature, without the need of high shear or neutralization. This includes the ability of the product to be post-added to an emulsion, e.g. in the case where it is desirable to convert a thin lotion to a thick cream. It is able to do this without the product becoming sticky, slimy, stringy, jelly-like. High levels can be used in formulations containing 1,3-propanediol without noticeable flaking of the emulsion during rub-out on the skin.

Advantageously it has been found by the inventors that modified starch of the type “2” is particularly useful as a film former in compositions of the invention, or as a thickener when used in higher concentrations. Modified starch of the type “b” has particularly been found to be useful as a fixative (e.g. in hair set compositions such as hair gels). It can provide emulsion stability and foam stability to compositions As such, this modified starch has been found to be able to replace sulfate surfactants in personal care compositions. Addition of the material can be done without high shear mixing at room or elevated temperature, needs no neutralization, and can be added in either the aqueous or oil phase, or added after an emulsion is formed. When modified starch “2” is used, the personal care compositions may thus comprise an absence of sulfate compounds.

Advantageously it has been found by the inventors that modified starch of the type “3” is particularly useful as it forms clear gels that are salt insensitive, present no stickiness in rub-out or after-feel, and have excellent rheological properties. Starch 3 was observed to improve composition flow properties, giving a predictable viscous behavior when dispensed. When starch 3 was incorporated into an exemplary shampoo composition, the composition flowed as expected when the fluid was rubbed into the wet scalp, generating a more stable foam that had lubricity to its feel. Starch 3 was observed to function as a suspending agent for conventional cleanser compositions, enabling a sulfate-free product that provides hair softening. Suspending properties of modified starch 3 are also beneficial in skin care formulations (e.g. color cosmetic formulations) where the starch can help suspend pigments, fillers, emulsifiers and emollients. Starch 3 may be added from about 0.1 to 99 weight percent by dry mass relative to the total dry mass weight of the composition, with higher amounts useful in gel formulations. Hair care formulations, such as shampoos, also benefit from the addition of modified starch according to the invention.

Advantageously it has been found by the inventors that modified starch of the type “4” is particularly useful as a film former in compositions of the invention. It has been found to be particularly useful in increasing water and wear resistance of compositions of the invention, and as a suspending agent. This starch has been found to reduce stickiness of compositions and so provides particularly desirable results when included in sunscreens and make up compositions. Examples of compositions that are deemed to particularly suitable for starch “4” based on these properties include: sun screens, moisturizing lotions, acne creams, mascara, lipsticks, foundations, various ointments, antiperspirants, etc.

Advantageously it has been found by the inventors that modified starch of the type “5” is useful as thickener or gel former for personal care compositions according to the invention. Such modified starch has been found to be useful in providing clear gels and serums, and so is eminently suitable for use in eye creams and serums. No neutralization is needed, and it can be added without high shear to an aqueous phase in which lipophilic actives have already been solubilized. The gels have firm shape, picks up easily, exhibit an appealing peaking effect without stringiness, spread easily with no jelly-like character, and do not possess negative stickiness or sliminess after the product is applied. This product is an excellent alternative to synthetic acrylate-based thickeners. In embodiments, formulations containing starch 5 thus may be devoid of synthetic acrylate-based thickeners.

### 2. Specific Exemplary Compositions Comprising Starches of the Invention

### 2.1 Example Body Lotion Formulation

The following formulation was prepared using starches 1 and 2, which are as described above in table 1, and the lotion was then used in further lab evaluations. Sodium starch octenyl succinate, obtained commercially from Aqdot® (Cambridge, UK) was also used in the formulation.

TABLE 2

| Phase | % | Material used | Description | Supplier | Function |
|---|---|---|---|---|---|
| A | 59.7% | Water | | | Solvent |
| B | 15.0% | Zemea ® | Bio-1,3-propanediol | DuPont Tate | Humectant |
| | | | | & Lyle | |
| B | 1.0% | sensiva ® PA 40 | Phenylpropanol (and) | Schuelke | Preservative |
| | | | Propanediol (and) | | |
| | | | Caprylyl Glycol (and) | | |
| | | | Tocopherol | | |
| B | 1.5% | Aq ™Star | Sodium starch octenyl | Aqdot | Emulsifier |
| | | | succinate | | |
| B | 2.0% | Starch 1 - | Hydroxypropyl starch | Tate & Lyle | Polymer |
| | | TEXTURLUX ® | phosphate | | |
| | | Rheo | (crosslinked) | | |
| B | 0.3% | Keltrol ® CG | Xanthan Gum | CPKelco | Polymer |
| C | 15.0% | LexFeel ® Natural | Heptyl Undecylenate | Inolex | Emollient |
| C | 2.5% | Crodacol ™ C70 | Cetyl Alcohol | Croda | Thickener |
| C | 1.5% | Aq ™Star | Sodium starch octenyl | Aqdot | Emulsifier |
| | | | succinate | | |
| C | 0.5% | Esterlac ™ Care+ | Sodium Isostearoyl | Corbion | Emulsifier |
| | | | Lactylate | | |
| C | 1.0% | Starch 2 - | Hydrolyzed Corn | Tate & Lyle | Polymer |
| | | TEXTURLUX ® | Starch Hydroxyethyl | | |
| | | Stabil | Ether | | |

The lotion was prepared by first preheating the water “A” to 80° C. Components “B” were premixed then added to A with propeller stirring. The resulting mixture was heated to 75° C. followed by addition of components “C” with propeller stirring. The mixture may be optionally homogenized until glossy if needed. The lotion was then evaluated for tactile performance. The lotion may be administered by any suitable method, such as by pump or tube.

### Performance Summary

The example lotion formulation prepared as described above was applied to the skin of a human subject and was found to smooth easily on the skin without running or dripping. The product was found to be excellent in sealing moisture and so would be eminently suitable as a moisturizer, particularly for use post-bathing (e.g. after showering).

Advantageously, the lotion was found to have advantage for persons with darker complexions by not producing an “ashy” appearance. An “ashy appearance” is typical of some conventional personal care items that are prone to leaving a chalky residue on the skin following application.

The rheology of the formulation offers shear thinning behavior with a slight degree of elasticity. This behavior is not only appealing aesthetically to potential consumers, but enables films to be produced on the skin that are relatively thick yet consistent. This is particularly advantageous for the utilization of cosmetic actives.

By use of modified starch and propan-1,3-diol in the formulation, it was possible to provide the beneficial tactile properties mentioned above in an absence of hydrophilic surfactants. This has the advantage that disruption of the barrier lipid bilayers of the skin (an effect that may be caused by conventional surfactants) is avoided.

The combination of modified starch in the formulation with propan-1,3-diol also was found to provide moisturisation without stickiness, and also excellent solvency properties. This thus makes personal care compositions of the invention useful for carrying a wide range of aqueous-based cosmetic actives.

### 2.2 Example Anti-Wrinkle Eye Gel Composition

The following gel formulation was prepared using starch 5 which is as described above in table 1, and the gel was then used in further lab evaluations.

TABLE 3

| Phase | % | Tradename | Description (e.g., INCI) | Supplier | Function |
|---|---|---|---|---|---|
| A | 74.9% | Water | — | — | Solvent |
| B | 15.0% | Zemea ® | Propanediol | DuPont Tate & | Humectant |
| | | | | Lyle | |
| B | 2.0% | Tween ® 80 | Polysorbate 80 | Croda | Emulsifier |
| | | | | | (solubiliser) |
| B | 1.5% | sensiva ® PA 40 | Phenylpropanol (and) | Schülke | Preservative |
| | | | Propanediol (and) | | |
| | | | Caprylyl Glycol (and) | | |
| | | | Tocopherol | | |
| B | 0.1% | Eastman | Retinyl Linoleate | Eastman | Active |
| | | GEM ™ Retinyl | | | |
| | | Linoleate | | | |
| C | 6.5% | Starch 1 - | Hydroxypropyl starch | Tate & Lyle | Polymer |
| | | TEXTURLUX ® | phosphate (crosslinked) | | |
| | | Feel | | | |

The gel was prepared by premixing components B with water “A” with minimal stirring. Starch “C” was then added with propeller stirring or side sweep until the mixture formed a clear gel. The gel was then evaluated for tactile performance.

### Performance Summary

The example gel formulation prepared as described above was applied to the skin of a human subject and was found to pick up and spread easily without stickiness, stringiness, or sliminess.

The modified starch of the invention provided as component C was found to integrate well with the rest of the formulation to form a cosmetically-appealing gel that has a high degree of bio-based content. The combination with 1,3-propanediol was in particular found to provide improved moisturisation, and excellent aesthetic appearance during Pick-up, Rub-out, and After-feel. Moreover, it was found that use of the starch of the invention aided in reducing the amount of polysorbate 80 as solubiliser required to provide a dear composition with the lipophilic active ingredient retinyl linoleate.

### 2.3 Example Facial Lotion

The following formulation was prepared using starches 1, 2 and 4, which are as described above in table 1, and the formulation was then used in further lab evaluations.

TABLE 4

| Phase | % | Tradename | Description (e.g., INCI) | Supplier | Function |
|---|---|---|---|---|---|
| A | 40.5% | Water | | | Solvent |
| B | 15.0% | Zemea ® | Propanediol | DuPont | Humectant |
| | | | | Tate & Lyle | |
| B | 1.5% | sensiva ® PA 40 | Phenylpropanol (and) | Schülke | Preservative |
| | | | Propanediol (and) Caprylyl | | |
| | | | Glycol (and) Tocopherol | | |
| B | 1.0% | Starch 2 - | Hydrolyzed Corn Starch | Tate & Lyle | Polymer |
| | | TEXTURLUX ® | Hydroxyethyl Ether | | |
| | | Stabil | | | |
| B | 1.0% | Starch 1 - | Hydroxypropyl starch | Tate & Lyle | Polymer |
| | | TEXTURLUX ® | phosphate (crosslinked) | | |
| | | Rheo | | | |
| B | 1.0% | Starch 4 - | Acid/enzyme thinned starch - | Tate & Lyle | Polymer |
| | | TEXTURLUX ® | Maltodextrin | | |
| | | Resist | | | |
| C | 15.0% | Crodamol ™ | Isostearyl Isostearate | Croda | Emollient |
| | | ISIS | | | |
| C | 5.0% | Arlacel ™ 165 | Glyceryl Stearate (and) PEG- | Croda | Emulsifier |
| | | | 100 Stearate | | |
| C | 1.0% | Myvatex ™ P28 | Glyceryl Stearate (and) | Kerry | Emulsifier |
| | | XL K | Sodium Stearoyl Lactylate | | |

The lotion was prepared by first preheating the water “A” to 60° C. Components “B” were first premixed then added to A with propeller stirring until a uniform mixture was obtained. Components “C” were mixed and heated to 60° C. before being added to the mixture of A/B with propeller stirring. The mixture was homogenized until glossy. The lotion was then evaluated for tactile performance.

### Performance Summary

The lotion provided a highly desirable aesthetic appearance and was found to have a pleasant feel. The lotion was found to provide excellent moisturisation and displayed substantial water resistance. Optionally, the emollient component may be replaced with an organic sunscreen active to provide sun protection, such as for a daily moisturizing lotion providing sun protection. The specific combination of starches provided was observed to enhance emulsion stabilization, yield value and film forming activity. The combination of the starches with bio-1,3-propanediol was found to add humectancy and allow for rapid introduction of the starches. This combination also enhanced the preservation of the lotion and was able to improve the ability of the formulation to deliver hydrophilic active agents.

### 2.4 Example Anti-Wrinkle Eye Serum

The following formulation was prepared using starch 1, which is as described above in table 1, and the formulation was then used in further lab evaluations.

TABLE 5

| Phase | % | Tradename | Description (e.g., INCI) | Supplier | Function |
|---|---|---|---|---|---|
| A | 76.5% | Water | | | Solvent |
| B | 15.0% | Zemea ® | Propanediol | DuPont | Humectant |
| | | | | Tate & Lyle | |
| B | 2.0% | Tween ® 80 | Polysorbate 80 | Croda | Emulsifier |
| | | | | | (solubiliser) |
| B | 1.5% | sensiva ® PA | Phenylpropanol (and) | Schülke | Preservative |
| | | 40 | Propanediol (and) Caprylyl | | |
| | | | Glycol (and) Tocopherol | | |
| B | 0.1% | Eastman | Retinyl Linoleate | Eastman | Active |
| | | GEM ™ Retinyl | | | |
| | | Linoleate | | | |
| C | 5.0% | Starch 1 - | Hydroxypropyl starch | Tate & Lyle | Polymer |
| | | TEXTURLUX ® | phosphate (crosslinked) | | |
| | | Rheo | | | |

The serum was prepared by premixing components B and then adding to water “A” with minimal stirring. Starch “C” was then added with propeller stirring or side sweep until the mixture formed a clear gel. The gel was then evaluated for tactile performance.

### Performance Summary

This formulation was found to provide an exceptionally clear serum. The serum was found to dispense easily from a narrow orifice, but did not run when placed on a finger or applicator. The serum spreads easily without stickiness, stringiness, or sliminess.

The modified starch of the invention acted as a rheology modifier which integrated easily with the other formulation components. The use of bio-1,3-propanediol in addition to the modified starch of the invention in particular was found to provide improved moisturisation, and excellent aesthetic appearance during Pick-up, Rub-out, and After-feel. Moreover, it was found that use of the starch of the invention aided in reducing the amount of polysorbate 80 as solubiliser required to provide a clear composition with the lipophilic active ingredient retinyl linoleate.

### 2.5 Example Hand Cream

The following formulation was prepared using starches 1 and 2, which are as described above in table 1, and the formulation was then used in further lab evaluations.

TABLE 6

| Phase | % | Tradename | Description (e.g., INCI) | Supplier | Function |
|---|---|---|---|---|---|
| A | 58.0% | Water | | | Solvent |
| B | 15.0% | Zemea ® | Propanediol | DuPont Tate | Humectant |
| | | | | & Lyle | |
| B | 1.0% | sensiva ® PA 40 | Phenylpropanol (and) | Schülke | Preservative |
| | | | Propanediol (and) Caprylyl | | |
| | | | Glycol (and) Tocopherol | | |
| B | 1.5% | Aq ™Star M1 | Sodium Starch Octenyl | Aqdot | Emulsifier |
| | | | Succinate (and) Cucurbituril | | |
| B | 3.0% | Starch 1 - | Hydroxypropyl starch | Tate & Lyle | Polymer |
| | | TEXTURLUX ® | phosphate (crosslinked) | | |
| | | Rheo | | | |
| C | 15.0% | Myvacet ™ 9- | Acetylated Hydrogenated | Kerry | Emollient |
| | | 45K | Vegetable Glyceride | | |
| C | 2.5% | Crodacol ™ C70 | Cetyl Alcohol | Croda | Thickener |
| C | 1.5% | Aq ™Star M1 | Sodium Starch Octenyl | Aqdot | Emulsifier |
| | | | Succinate (and) Cucurbituril | | |
| C | 0.5% | Esterlac ™ | Sodium Isostearoyl Lactylate | Corbion | Emulsifier |
| | | Care+ | | | |
| C | 1.0% | Starch 2 - | Hydrolyzed Corn Starch | Tate & Lyle | Polymer |
| | | TEXTURLUX ® | Hydroxyethyl Ether | | |
| | | Hold | | | |
| D | 1.0% | Starch 1 - | Hydroxypropyl starch | Tate & Lyle | Polymer |
| | | TEXTURLUX ® | phosphate (crosslinked) | | |
| | | Rheo | | | |

The cream was prepared by first preheating the water “A” to 80° C. The AqStar component was then added with stirring until clear. The remainder of components “B” were then premixed and added to A with stirring. The resulting mixture was heated to 75° C. Components “C” were mixed and added to the mixture of A/B with stirring. The mixture may at this stage be optionally homogenized until glossy. D was added to the mixture of A to C with stirring. The resulting cream was then evaluated for tactile performance.

### Performance Summary

The cream was found to be firm and dispensable from a tube or pump. Although the formulation is relative thick, the formulation was found to spread easily on the skin, thus displaying desirable film forming properties. It was found that the film left on the skin was described as feeling protective, but not greasy, sticky, or tacky.

It was found that the use of the modified corn starches of the invention enhanced stabilization of the emulsion. The use of starch 1 in particular was found to add viscosity to the mixture, thus turning a lotion formulation into a thick cream. It was observed that the cream displayed no stickiness, stringiness, sliminess, or jelly-like character.

The rheology of the formulation offers shear thinning behavior with a slight degree of elasticity. This behavior is not only appealing aesthetically to potential consumers, but enables films to be produced on the skin that are relatively thick yet consistent. This is particularly advantageous for the utilization of cosmetic actives.

By using of modified starch and propan-1,3-diol in the formulation, it was possible to provide the beneficial tactile properties mentioned above in an absence of hydrophilic surfactants. This has the advantage that disruption of the barrier lipid bilayers of the skin (an effect that may be caused by conventional surfactants) is avoided, and resistance to wash-off is excellent, thus improving water resistance. The combination of modified starch in the formulation with propan-1,3-diol also was found to provide moisturisation without stickiness, and also excellent solvency properties. This thus makes certain embodiments of personal care compositions of the invention useful for carrying a wide range of aqueous-based cosmetic actives.

### 2.6 Example Rejuvenating Eye Gel

The following formulation was prepared using TEXTURLUX™ Feel brand hydroxypropyl starch phosphate, which is as described above in table 1.

TABLE 7

| Phase | % | Tradename | Description (e.g., INCI) | Supplier | Function |
|---|---|---|---|---|---|
| A | 75.35% | Water | Water | | Solvent |
| B | 15.0% | Zemea ® | Propanediol | DuPont Tate | Humectant |
| | | | | & Lyle | |
| B | 1.0% | Spectrastat ™ | Caprylhydroxamic Acid (and) | Inolex | Preservative |
| | | G2 | Glyceryl Caprylate (and) | | |
| | | | Glycerin | | |
| B | 1.0% | Tween ® 80 | Polysorbate 80 | Croda | Surfactant |
| B | 0.5% | PURAC ® BF | Water (and) Lactic Acid (and) | Corbion | Active |
| | | P/41 | Potassium Lactate | | |
| B | 0.2% | PhytoTrace ™ | Olea Europaea (Olive) Leaf | Inolex | Active |
| | | Olive | Extract (and) Glycerin (and) | | |
| | | | Water | | |
| B | 6.5% | Starch 5 - | Hydroxypropyl starch | Tate & Lyle | Polymer |
| | | TEXTURLUX ® | phosphate (crosslinked) | | |
| | | Feel | | | |
| B | 0.45% | Orchilda Eye | Fragrance | Orchidia | Fragrance |
| | | Cream | | | |
| | | Fragrance | | | |

The cream is prepared by premixing the “B” components until uniform, then adding the premixed “B” components to the water “A” with slow stirring until uniform and clear. It provides moisturization, gentle pH correction and invigoration for tired eyes, and helps reduce redness and puffiness while providing intense hydration.

### 2.7 Example Daily Dose Sunscreen

The following formulation was prepared using TEXTURLUX™ Rheo brand hydroxypropyl starch phosphate and TEXTURLUX™ Resist brand maltodextrin, which are as described above in table 1.

TABLE 8

| Phase | % | Tradename | Description (e.g., INCI) | Supplier | Function |
|---|---|---|---|---|---|
| A | 35.0% | Water | Water | | Solvent |
| B | 4.0% | ESTERLAC ™ | Sodium Behenoyl Lactylate | Corbion | Emulsifier |
| | | Perform | | | |
| B | 2.0% | Crodacol ™ | Cetearyl Alcohol | Corbion | Wax |
| | | C70 | | | |
| C | 10.0% | Zemea ® | Propanediol | DuPont Tate | Humectant |
| | | | | & Lyle | |
| C | 1.0% | Spectrastat ™ | Caprylhydroxamic Acid (and) | Inolex | Preservative |
| | | G2 | Glyceryl Caprylate (and) | | |
| | | | Glycerin | | |
| C | 5.0% | Solaveil ™ CT- | Caprylic/Capric Triglyceride | Croda | Active |
| | | 12W | (and) Titanium Dioxide (and) | | |
| | | | Polyhydroxystearic Acid | | |
| | | | (and) Aluminum Stearate | | |
| | | | (and) Alumina | | |
| C | 2.0% | Starch 1 - | Hydroxypropyl starch | Tate & Lyle | Polymer |
| | | TEXTURLUX ® | phosphate (crosslinked) | | |
| | | Rheo | | | |
| C | 1.0% | Starch 4 - | Acid/enzyme thinned starch - | Tate & Lyle | Film Former |
| | | TEXTURLUX ® | Maltodextrin | | |
| | | Resist | | | |
| D | 15.0% | Solaveil ™ CT- | Caprylic/Capric Triglyceride | Croda | Active |
| | | 300 | (and) Titanium Dioxide (and) | | |
| | | | Polyhydroxystearic Acid | | |
| | | | (and) Aluminum Stearate | | |
| | | | (and) Alumina | | |
| D | 5.0% | Solaveil ™ XT- | Titanium Dioxide (and) | Croda | Active |
| | | 300 | Caprylic/Capric Triglyceride | | |
| | | | (and) Polyhydroxystearic | | |
| | | | Acid (and) Stearic Acid (and) | | |
| | | | Alumina | | |
| D | 15.0% | LexFeel ® | Triheptanoin (and) C13-16 | Inolex | Emollient |
| | | WOW | Isoparaffin | | |
| D | 5.0% | LexFilm ® Sun | Capryloyl Glycerin/Sebacic | Inolex | Film Former |
| | | Natural | Acid Copolymer | | |

The cream is prepared by first heating the water “A” to 85° C. While maintaining temperature at 85° C., the “B” components are added to the water “A” with propeller mixing until melted and uniform. The “C” components are premixed then added to the mixed “A” and “B” components with propeller mixing at 85° C. until uniform. While maintaining mixing, heat is removed. When the temperature of the “A”/“B”/“C” mixture reaches 60° C., the premixed “D” components are added with propeller mixing until the mixture is uniform and glossy. The cream provides daily sun protection together with beneficial skin protection.

### 2.8 Example Nourishing Night Cream

The following formulation was prepared using TEXTURLUX™ Rheo brand hydroxypropyl starch phosphate, which is as described above in Table 1.

TABLE 9

| Phase | % | Tradename | Description (e.g., INCI) | Supplier | Function |
|---|---|---|---|---|---|
| A | 84.7% | Water | Water | | Solvent |
| B | 4.0% | ESTERLAC ™ | Sodium Behenoyl Lactylate | Corbion | Emulsifier |
| | | Perform | | | |
| B | 2.0% | SPAN 60 | Sorbitan Stearate | Croda | Emulsifier |
| C | 5.0% | Zemea ® | Propanediol | DuPont Tate | Humectant |
| | | | | & Lyle | |
| C | 1.0% | Spectrastat ™ | Caprylhydroxamic Acid | Inolex | Preservative |
| | | G2 | (and) Glyceryl Caprylate | | |
| | | | (and) Glycerin | | |
| C | 5.0% | Starch 1 - | Hydroxypropyl starch | Tate & Lyle | Polymer |
| | | TEXTURLUX ™ | phosphate (crosslinked) | | |
| | | Rheo | | | |
| C | 0.2% | PhytoTrace ™ | Ficus Carcia (Fig) Fruit | Inolex | Active |
| | | Fig | Extract (and) Glycerin (and) | | |
| | | | Water | | |
| D | 15.0% | SustOleo ™ | Triheptanoin | Inolex | Emollient |
| | | MCT | | | |

The cream is prepared by premixing the “B” components then adding to the water “A” with propeller mixing until uniform. The “C” components are premixed, then the “A”/“B” mixture is added to the premixed “C” components with fast propeller stirring. The mixture is homogenized if necessary to provide a glossy cream. The cream can be used overnight to help the recovery of facial skin.

### 2.9 Example Moisturizing Spray Lotion

The following formulation was prepared using TEXTURLUX™ Stabil brand hydrolyzed corn starch hydroxyethyl ether, which is as described above in table 1.

TABLE 10

| Phase | % | Tradename | Description (e.g., INCI) | Supplier | Function |
|---|---|---|---|---|---|
| A | 71.5% | Water | Water | | Solvent |
| B | 10.0% | Zemea ® | Propanediol | DuPont Tate | Humectant |
| | | | | & Lyle | |
| B | 1.0% | Spectrastat ™ G2 | Caprylhydroxamic Acid (and) | Inolex | Preservative |
| | | | Glyceryl Caprylate (and) | | |
| | | | Glycerin | | |
| B | 1.0% | Purasal ® | Water (and) Sodium Lactate | Corbion | Active |
| | | MOISTXS | (and) Sodium Gluconate | | |
| B | 1.0% | Safic Care T GXC | Cyampois Tetragonolobus | Safic Alcan | Polymer |
| | | | (Guar) Gum (and) Xanthan | | |
| | | | Gum | | |
| C | 0.5% | LexFeel ® Natural | Triheptanoin | Inolex | Emollient |
| C | 1.0% | Starch 2 - | Hydrolyzed Corn Starch | Tate & Lyle | Polymer |
| | | TEXTURLUX ™ | Hydroxyethyl Ether | | |
| | | Stabil | | | |

The spray is prepared by premixing the “B” components then adding to the water “A” with propeller mixing until uniform. The “C” components are premixed, then added to the “A”/“B” mixture with propeller stirring until uniform. The mixture is homogenized briefly to glossiness, then propeller stirred until smooth. The spray can be used as post-shower protection to provide hydration and to give skin a soft, moisturized look and feel.

### 2.10 Example Shampoo

The following formulation was prepared using TEXTURLUX™ Hold brand hydroxypropyl starch phosphate, which is as described above in table 1.

TABLE 11

| Phase | % | Tradename | Description (e.g., INCI) | Supplier | Function |
|---|---|---|---|---|---|
| A | 45.54% | Water | Water | | Solvent |
| B | 15.0% | Lexaine ® C | Cocamidopropyl Betaine | Inolex | Surfactant |
| B | 30.0% | Crodasinic ™ | Sodium Lauroyl Sarcosinate | Croda | Surfactant |
| | | LS30 | | | |
| C | 5.0% | Zemea ® | Propanediol | DuPont Tate | Humectant |
| | | | | & Lyle | |
| C | 3.0% | ESTERLAC ™ | Sodium Isostearoyl Lactylate | Corbion | Surfactant |
| | | Care+ | | | |
| C | 1.0% | Spectrastat ™ G2 | Caprylhydroxamic Acid (and) | Inolex | Preservative |
| | | | Glyceryl Caprylate (and) | | |
| | | | Glycerin | | |
| C | 0.1% | Starch 3 - | Hydroxypropyl starch | Tate & Lyle | Polymer |
| | | TEXTURLUX ™ | phosphate (crosslinked) | | |
| | | Hold | | | |
| C | 0.19% | Fragrance | Kerry Lemon Oil Cold | Kerry | Fragrance |
| | | | Pressed | | |
| C | 0.17% | Fragrance | Kerry Lavender Oil | Kerry | Fragrance |
| D | q.s. | PURAC(R) | Lactic Acid | Corbion | Neutralizer |
| | | HiPure 90 | | | |
| E | q.s. | PURASAL(R) | Potassium Lactate | Corbion | Salt |
| | | HiPure 90 | | | |

The shampoo is prepared by adding the “B” components to the water “A” with slow sweep stirring. The “C” components are premixed, then added to the “A”/“B” mixture with stirring until uniform. The “D” component is added as necessary until a pH of about 5.5 is reached, then the “E” component is added to provide the desired viscosity. The shampoo is clear and sulfate-free and provides good flow, foam, feel, finish and fragrance.

It will be appreciated that numerous modifications to the described embodiments may be made without departing from the scope of the invention, as defined in the appended claims.

The described and illustrated embodiments are to be considered as illustrative and not restrictive in character, it being understood that only the preferred embodiments have been shown and described and that all changes and modifications that come within the scope of the invention, as defined in the appended claims, are desired to be protected.

It should be understood that while the use of words such as “preferable”, “preferably”, “preferred” or “more preferred” in the description suggest that a feature so described may be desirable, it may nevertheless not be necessary and embodiments lacking such a feature may be contemplated as within the scope of the invention as defined in the appended claims. In relation to the claims, it is intended that when words such as “a,” “an,” “at least one,” or “at least one portion” are used to preface a feature there is no intention to limit the claim to only one such feature unless specifically stated as such in the claim. When the language “at least a portion” and/or “a portion” is used the item can include a portion and/or the entire item unless specifically stated to the contrary. Throughout this specification, the term “comprising” or “comprises” means including the component(s) specified but not to the exclusion of the presence of others.

Various additional embodiments of the disclosure are provided by the following enumerated embodiments, which can be combined in any number and in any combination that is not technically or logically inconsistent.

### Embodiment 1

A personal care composition comprising:
a propanediol or an ester thereof; and
a modified starch selected from the group consisting of modified corn starch such as modified dent corn starch or modified waxy corn starch; modified wheat starch; modified barley starch; modified tapioca starch; modified triticale starch; modified sorghum starch; modified rice starch; and modified legume starch such as modified pea starch or modified soybean starch.

### Embodiment 2

The personal care composition according to Embodiment 1 wherein the propanediol is 1,3-propanediol.

### Embodiment 3

The personal care composition according to Embodiment 2 wherein the 1,3-propanediol is biologically derived 1,3-propanediol (i.e. bio-PDO), optionally derived by fermentation methods.

### Embodiment 4

The personal care composition according to any preceding Embodiment comprising more than one modified starch.

### Embodiment 5

The personal care composition according to any preceding Embodiment wherein the modified starch is selected from the group consisting of a hydrolysed starch, an oxidized starch, an esterified starch, an etherified starch, an acetylated starch, a C₁₋₁₀alkylated starch, a hydroxyC₁₋₁₀alkylated starch, a crosslinked starch, a phosphorylated starch, a succinylated starch, and a starch that contains two or more of said modifications.

### Embodiment 6

The personal care composition according to any preceding Embodiment wherein the modified starch is selected from the group consisting of modified dent corn starch, modified waxy corn starch and modified tapioca starch.

### Embodiment 7

The personal care composition according to any preceding Embodiment wherein the at least one modified starch used in the composition is annealed, extruded, drum dried, spray-dried, recrystallized, and/or agglomerated.

### Embodiment 8

The personal care composition according to Embodiment 5 or 6 wherein the hydrolysed starch is enzymatically hydrolysed.

### Embodiment 9

The personal care composition according to any one of Embodiments 5 to 8 wherein the hydrolysed starch is maltodextrin.

### Embodiment 10

The personal care composition according to any one of Embodiments 5 to 7 wherein the at least one modified starch is hydroxypropylated or hydroxyethylated starch.

### Embodiment 11

The personal care composition according to any preceding Embodiment wherein the starch is cross-linked, preferably by phosphorylation.

### Embodiment 12

The personal care composition according to any preceding Embodiment wherein the personal care composition is a fluid (e.g. liquid) composition.

### Embodiment 13

The personal care composition according to Embodiment 12 wherein the fluid composition is selected from the group consisting of a gel, a cream, a solution, a spray, a lotion, an ointment, an emulsion, a mousse, a cleanser, a slurry, a serum, or a paste.

### Embodiment 14

The personal care composition according to any one of Embodiments 1 to 13 wherein the personal care composition is a cosmetic composition, a dermatological care composition, or a body care compositions.

### Embodiment 15

The personal care composition according to any one of Embodiments 1 to 6 or 10 to 14 wherein:
the personal care composition is an emulsion;
the propanediol is 1,3-propanediol; and
the modified starch is crosslinked (e.g., phosphorylated) and hydroxypropylated;
optionally wherein the composition does not contain carbomer and/or Xanthan Gum.

### Embodiment 16

The personal care composition according to any one of Embodiments 1 to 6 or 10 to 14, wherein:
the personal care composition is a cleanser, optionally a foaming cleanser;
the propanediol is 1,3-propanediol; and
the modified starch is crosslinked (e.g., phosphorylated) and hydroxypropylated.

### Embodiment 17

The personal care composition according to any one of Embodiments 1 to 6 or 10 to 14, wherein:
the personal care composition is a gel or serum;
the propanediol is 1,3-propanediol; and
the modified starch is crosslinked (e.g., phosphorylated) and hydroxypropylated.

### Embodiment 18

The personal care composition according to any one of Embodiments 1 to 6 or 10 to 14, wherein:
the personal care composition is a cream, preferably an eye cream;
the propanediol is 1,3-propanediol; and
the modified starch is crosslinked (e.g., phosphorylated).

### Embodiment 19

The personal care composition according to any one of Embodiments 15 to 18, wherein the modified starch is modified waxy corn starch.

### Embodiment 20

The personal care composition according to any one of Embodiments 1 to 6 or 10 to 14, wherein:
the personal care composition is an emulsion;
the propanediol is 1,3-propanediol; and
the modified starch is hydroxyethylated.

### Embodiment 21

The personal care composition according to Embodiment 20, wherein the modified starch is modified dent corn starch.

### Embodiment 22

The personal care composition according to any one of Embodiments 1 to 9, wherein:
the personal care composition is a sunscreen or makeup;
the propanediol is 1,3-propanediol; and
the modified starch is maltodextrin.

### Embodiment 23

The personal care composition according to any one of Embodiments 1 to 9, or Embodiments 12 to 14 when dependent on Embodiments 1 to 9 wherein:
the personal care composition is a film forming composition;
the propanediol is 1,3-propanediol; and
the modified starch is maltodextrin.

### Embodiment 24

The personal care composition according to any preceding Embodiment wherein the composition comprises up to about 50% wt of the modified starch by dry mass relative to the dry mass weight of the personal care composition, optionally from about 1-30%, such as from about 4 to 7% wt or from 10-30% wt by dry mass, e.g. from about 12-28% wt by dry mass.

### Embodiment 25

The personal care composition according to any preceding Embodiment wherein the composition comprises up to about 80% wt of propanediol or ester thereof by dry mass relative to the dry mass weight of the personal care composition, optionally from about 10-65%, such as from about 20-65% wt by dry mass.

### Embodiment 26

The personal care composition according to any preceding Embodiment wherein the weight ratio of propanediol or ester thereof to modified starch in the composition is from 1:10 to 10:1, optionally from 1:5 to 8:1, and such as from about 1:1 to 6:1, preferably from 1.5:1 to 6:1, such as about 5:1.

### Embodiment 27

The personal care composition according to any preceding Embodiment wherein the composition further comprises one or more of the group consisting of: a carrier, a preservative, a therapeutic agent, a nutraceutical agent, an abrasive, an adsorbent, a stabilizer, a colourant, a thickener, a gellant, a solidifying agent, a structuring agent, an anti-foaming agent, a binder, a film former, a humectant, an essential oil, a filler, a surfactant, a plasticiser, an elasticiser, a conditioning agent, an emollient, an astringent, an antiperspirant compound, a sunscreen, a UV absorber, a fragrance, and a dispersant.

### Embodiment 28

The personal care composition according to any preceding Embodiment wherein the modified starch is a thickening starch and the personal care composition does not further comprise an additional thickening agent.

### Embodiment 29

The personal care composition according to any preceding Embodiment wherein the modified starch is a gelling starch and the personal care composition does not further comprise an additional gelling agent.

### Embodiment 30

The personal care composition according to any preceding Embodiment wherein the personal care composition does not further comprise xanthan gum; and/or an acrylate polymer; and/or a carbomer; and/or a hydrophilic surfactant; and/or a polymer selected from the group consisting of polyvinyl alcohol and vinylpyrollidone polymers; and/or a surfactant; and/or an emulsifier.

### Embodiment 31

The personal care composition according to Embodiment 1, wherein the personal care composition is a lotion, the lotion comprising:
from about 10-20% by weight bio-1,3-propanediol; and
from about 2 to 5% by weight of a modified corn starch selected from one or more of hydroxypropylated and crosslinked (e.g., phosphorylated) waxy corn starch, and hydroxyethylated dent corn starch; and
optionally one or more of the group consisting of emulsifiers, emollients, solvents and thickeners, wherein the weight provided is % wt based on the total weight of the composition.

### Embodiment 32

The personal care lotion according to Embodiment 31, wherein the lotion is an aqueous lotion, the lotion comprising:
from about 10-20% by weight bio-1,3-propanediol; and
from about 1 to 3% by weight of a hydroxypropylated and crosslinked (e.g., phosphorylated) waxy corn starch; and
from about 0.5 to 2% by weight of a hydroxyethylated dent corn starch; and
optionally one or more of the group consisting of emulsifiers, emollients, solvents and thickeners.

### Embodiment 33

The personal care lotion according to Embodiment 32, wherein the lotion is an aqueous lotion, the lotion comprising:
from about 10-20% by weight bio-1,3-propanediol; and
from about 1 to 3% by weight of a hydroxypropylated and crosslinked (e.g., phosphorylated) waxy corn starch; and
from about 0.5 to 2% by weight of a hydroxyethylated dent corn starch; and
from about 2 to 4% of an octenyl succinylated corn starch; and
from about 3 to 5% emulsifier; and
from about 2 to 3% thickener, such as cetyl alcohol; and
from about 12 to 18% emollient such as heptyl undecylenate; and
from about 0.5 to 2% preservative.

### Embodiment 34

The personal care composition according to any previous Embodiment wherein the modified corn starch and propanediol are dispersed or dissolved in the composition in a carrier medium, preferably dissolved.

### Embodiment 35

Use of a modified starch as a rheology modifier, thickener, gelling agent, film forming agent, emulsion stabilising agent, or foam stabilising agent in a personal care composition, wherein the modified starch is selected from one or more of the following: a modified corn starch such as modified dent corn starch or modified waxy corn starch; modified wheat starch; modified barley starch; modified tapioca starch; modified triticale starch; modified sorghum starch; modified rice starch; and modified legume starch such as modified pea starch or modified soybean starch.

### Embodiment 36

The use according to Embodiment 35 wherein the modified starch is selected from the group consisting of consisting of modified dent corn starch, modified waxy corn starch and modified tapioca starch.

### Embodiment 37

The use according to Embodiment 35 or 36, wherein the modified starch is selected from the group consisting of a hydrolysed starch, an oxidized starch, an esterified starch, an etherified starch, an acetylated starch, a C₁₋₁₀alkylated starch, a hydroxyC₁₋₁₀alkylated starch, a crosslinked starch, a phosphorylated starch, a succinylated starch, and a starch that contains two or more of said modifications.

### Embodiment 38

The use according to any one of Embodiments 35 to 37, wherein the at least one modified starch is annealed, extruded, drum dried, spray-dried, recrystallized, and/or agglomerated.

### Embodiment 39

The use according to any one of Embodiments 37 and 38, wherein the hydrolysed starch is enzymatically hydrolysed.

### Embodiment 40

The use according to any one of Embodiments 37 to 39 wherein the hydrolysed starch is maltodextrin.

### Embodiment 41

The use according to any one of Embodiments 35 to 38 wherein the at least one modified starch is hydroxypropylated or hydroxyethylated.

### Embodiment 42

The use according to any one of Embodiments 35 to 41, wherein the starch is cross-linked, preferably by phosphorylation.

### Embodiment 43

The use according to any one of Embodiments 35 to 42, wherein the modified starch is selected from one or more of the following:
a) a waxy corn starch that is hydroxypropylated and cross-linked (e.g. by phosphorylation); and
b) a dent corn starch that is hydroxyethylated; and
c) a maltodextrin; and
d) a waxy corn starch that is cross-linked (e.g. by phosphorylation).

### Embodiment 44

The use according to Embodiment 43, wherein the use is as a rheology modifier in a personal care composition, and wherein the modified starch is a waxy corn starch that is hydroxypropylated and cross-linked (e.g. by phosphorylation).

### Embodiment 45

The use according to Embodiment 44 wherein the personal care composition is an emulsion or cleanser.

### Embodiment 46

The use according to Embodiment 43, wherein the use is as a thickener in a personal care composition, and wherein the modified starch is selected from one or more of the following:
a) a waxy corn starch that is hydroxypropylated and cross-linked (e.g. by phosphorylation); and
b) a dent corn starch that is hydroxyethylated; and
c) a waxy corn starch that is cross-linked (e.g. by phosphorylation).

### Embodiment 47

The use according to Embodiment 43, wherein the use is as a gelling agent in a personal care composition, and wherein the modified starch is selected from one or more of the following:
a) a waxy corn starch that is cross linked (e.g. by phosphorylation) and optionally hydroxypropylated.

### Embodiment 48

The use according to Embodiment 43, wherein the use is as a film forming agent in a personal care composition, and wherein the modified starch is selected from one or more of the following:
a) a dent corn starch that is hydroxyethylated; and
b) a maltodextrin.

### Embodiment 49

The use according to Embodiment 43, wherein the use is as an emulsion stabilising agent in a personal care composition, wherein the modified starch is a dent corn starch that is hydroxyethylated.

### Embodiment 50

The use according to anyone of Embodiment 43 wherein the use is as a foam stabilising agent in a personal care composition, optionally a cleansing composition, wherein the modified starch is a waxy corn starch that is hydroxypropylated and cross-linked (e.g. by phosphorylation).

### Embodiment 51

The use according to any one of Embodiments 35 to 50, wherein the use comprises adding the modified starch to a carrier medium.

### Embodiment 52

The use according to Embodiment 51, wherein the personal care composition comprises propanediol or an ester thereof.

### Embodiment 53

The use according to Embodiment 52, wherein the use comprises separately or simultaneously adding the modified starch and the propanediol or ester thereof to the carrier medium.

### Embodiment 54

The use according to Embodiment 51, 52 or 53, wherein the modified corn starch and, if present, the propanediol or ester thereof are dispersed or dissolved in the carrier medium, preferably dissolved.

### Embodiment 55

The use according to any one of Embodiments 52 to 54 wherein the propanediol is 1,3-propanediol.

### Embodiment 56

The use according to Embodiment 55, wherein the 1,3-propanediol is biologically derived 1,3-propanediol bio-PDO), optionally derived by fermentation methods.

### Embodiment 57

The use according to any one of Embodiments 35 to 56, wherein the personal care composition comprises up to about 50% wt of the modified starch by dry mass relative to the dry mass weight of the personal care composition, optionally from about 1-30%, such as from about 4 to 7% wt or from 10-30% wt by dry mass, e.g. from about 12-28% wt by dry mass.

### Embodiment 58

The use according to any one of Embodiments 52 to 57 wherein the personal care composition comprises up to about 80% wt of the propanediol or ester thereof by dry mass relative to the dry mass weight of the personal care composition, optionally from about 10-65%, such as from about 20-65% wt by dry mass.

### Embodiment 59

The use according to any one of Embodiments 52 to 58 wherein the weight ratio of propanediol or ester thereof to modified starch in the personal care composition is from 1:10 to 10:1, optionally from 1:5 to 8:1, and such as from about 1:1 to 6:1, such as from 1.5:1 to 6:1, e.g. about 5:1.

### Embodiment 60

The use according to any one of Embodiments 35 to 59, wherein the personal care composition further comprises one or more of the group consisting of: a preservative, a therapeutic agent, a nutraceutical agent, an abrasive, an adsorbent, a stabilizer, a colourant, a thickener, a gellant, a solidifying agent, a structuring agent, an anti-foaming agent, a binder, a film former, a humectant, an essential oil, a filler, a surfactant, a plasticiser, an elasticiser, a conditioning agent, an emollient, an astringent, an antiperspirant compound, a sunscreen, a UV absorber, a fragrance, and a dispersant.

### Embodiment 61

The use according to any one of Embodiments 35 to 46 or any one of Embodiments 51 to 60 when dependent on any of Embodiments 35 to 46, wherein the personal care composition does not comprise a thickener in addition to the modified starch.

### Embodiment 62

The use according to Embodiment 47 or any one of Embodiments 51 to 61 when dependent on Embodiment 47, wherein the personal care composition does not comprise a gelling agent in addition to the modified starch.

### Embodiment 63

The use according to any one of Embodiments 35 to 62, wherein the personal care composition does not comprise xanthan gum; and/or an acrylate polymer; and/or a carbomer and/or a hydrophilic surfactant; and/or a polymer selected from the group consisting of polyvinyl alcohol and vinylpyrollidone polymers; and/or a surfactant; and/or an emulsifier in addition to the modified starch.

### Embodiment 64

A method of modifying the rheological properties, thickness, gelling properties, film forming properties, emulsion stability or foam stability of a carrier in a personal care composition, the method comprising adding to the carrier.
a modified starch and optionally a propanediol or an ester thereof,
to form a personal care composition having respectively modified rheological properties, thickness, gelling properties, film forming properties, emulsion stability or foam stability properties relative to the composition prior to addition of the modified starch and optionally a propanediol or an ester thereof to the carrier, wherein the modified starch is selected from one or more of the following:
a modified corn starch such as modified dent corn starch or modified waxy corn starch; modified wheat starch; modified barley starch; modified tapioca starch; modified triticale starch; modified sorghum starch; modified rice starch; and modified legume starch such as modified pea starch or modified soybean starch.

### Embodiment 65

The method according to Embodiment 64 wherein the modified starch is selected from the group consisting of modified dent corn starch, modified waxy corn starch and modified tapioca starch.

### Embodiment 66

The method according to Embodiment 64 or 65, wherein the modified starch is selected from the group consisting of a hydrolysed starch, an oxidized starch, an esterified starch, an etherified starch, an acetylated starch, a C₁₋₁₀alkylated starch, a hydroxyC₁₋₁₀alkylated starch, a crosslinked starch, a phosphorylated starch, a succinylated starch, and a starch that contains two or more of said modifications.

### Embodiment 67

The method according to any one of Embodiments 64 to 66, wherein the at least one modified starch used in the composition is annealed, extruded, drum dried, spray-dried, recrystallized, and/or agglomerated.

### Embodiment 68

The method according to any one of Embodiments 64 to 67, wherein the hydrolysed starch is enzymatically hydrolysed.

### Embodiment 69

The method according to any one of Embodiments 64 to 68 wherein the hydrolysed starch is maltodextrin.

### Embodiment 70

The method according to any one of Embodiments 64 to 67 wherein the at least one modified starch is hydroxypropylated, hydroxyethylated or acetylated.

### Embodiment 71

The method according to any one of Embodiments 64 to 70, wherein the starch is cross-linked, preferably by phosphorylation.

### Embodiment 72

The method according to any one of Embodiments 64 to 71, wherein the modified starch is selected from one or more of the following:
a) a waxy corn starch that is hydroxypropylated and cross-linked (e.g. by phosphorylation); and
b) a dent corn starch that is hydroxyethylated; and
c) a maltodextrin; and
d) a waxy corn starch that is cross-linked (e.g. by phosphorylation).

### Embodiment 73

The method according to Embodiment 72, wherein the method is a method of modifying the rheological properties of the carrier, and wherein the modified starch is a waxy corn starch that is hydroxypropylated and cross-linked (e.g. by phosphorylation).

### Embodiment 74

The method according to Embodiment 73 wherein the personal care composition is an emulsion or cleanser.

### Embodiment 75

The method according to Embodiment 72, wherein the method is a method of modifying the thickness of the carrier, and wherein the modified starch is selected from one or more of the following:
a) a waxy corn starch that is hydroxypropylated and cross-linked (e.g. by phosphorylation); and
b) a dent corn starch that is hydroxyethylated; and
c) a waxy corn starch that is cross-linked (e.g. by phosphorylation).

### Embodiment 76

The method according to Embodiment 72, wherein the method is a method of modifying the gelling properties of the carrier, and wherein the modified starch is selected from one or more of the following:
a) a waxy corn starch that is cross-linked (e.g. by phosphorylation) and optionally hydroxypropylated.

### Embodiment 77

The method according to Embodiment 72, wherein the method is a method of modifying the film forming properties of the carrier, and wherein the modified starch is selected from one or more of the following:
a) a dent corn starch that is hydroxyethylated; and
b) a maltodextrin.

### Embodiment 78

The method according to Embodiment 72, wherein the method is a method of modifying the emulsion stability of the carrier, and the modified starch is a dent corn starch that is hydroxyethylated.

### Embodiment 79

The method according to Embodiment 72, wherein the method is a method of modifying the foam stability of the carrier, optionally wherein the personal care composition is a cleansing composition, and wherein the modified starch is a waxy corn starch that is hydroxypropylated and cross-linked (e.g. by phosphorylation).

### Embodiment 80

The method according to any one of Embodiments 64 to 79, wherein the personal care composition comprises propanediol or an ester thereof.

### Embodiment 81

The method according to Embodiment 80, wherein the use comprises separately or simultaneously adding the modified starch and propanediol or ester thereof to the carrier medium.

### Embodiment 82

The method according to any one of Embodiments 64 to 81, wherein the modified starch and, if present, the propanediol or ester thereof are dispersed or dissolved in the carrier medium, preferably dissolved.

### Embodiment 83

The method according to any one of Embodiments 64 to 82 wherein the propanediol is 1,3-propanediol.

### Embodiment 84

The method according to Embodiment 83, wherein the 1,3-propanediol is biologically derived 1,3-propanediol bio-PDO), optionally derived by fermentation methods.

### Embodiment 85

The method according to any one of Embodiments 64 to 84, wherein, after addition of the modified starch, the personal care composition comprises up to about 50% wt of the modified starch by dry mass relative to the dry mass weight of the personal care composition, optionally from about 1-30%, such as from about 4 to 7% wt or from 10-30% wt by dry mass, e.g. from about 12-28% wt by dry mass.

### Embodiment 86

The method according to any one of Embodiments 80 to 85 wherein the propanediol or ester thereof in the final personal care composition comprises up to about 80% wt of the propanediol or ester thereof by dry mass relative to the dry mass weight of the personal care composition, optionally from about 10-65%, such as from about 20-65% wt by dry mass.

### Embodiment 87

The method according to any one of Embodiments 80 to 86 wherein the weight ratio of propanediol or ester thereof to modified starch in the personal care composition is from 1:10 to 10:1, optionally from 1:5 to 8:1, and such as from about 1:1 to 6:1, preferably from 1.5:1 to 6:1, such as about 5:1.

### Embodiment 88

The method according to any one of Embodiments 64 to 87, wherein the personal care composition further comprises one or more of the group consisting of: a preservative, a therapeutic agent, a nutraceutical agent, an abrasive, an adsorbent, a stabilizer, a colourant, a thickener, a gellant, a solidifying agent, a structuring agent, an anti-foaming agent, a binder, a film former, a humectant, an essential oil, a filler, a surfactant, a plasticiser, an elasticiser, a conditioning agent, an emollient, an astringent, an antiperspirant compound, a sunscreen, a UV absorber, a fragrance, and a dispersant.

### Embodiment 89

The method according to any one of Embodiments 64 to 88, wherein the personal care composition does not comprise xanthan gum; and/or an acrylate polymer; and/or a carbomer; and/or a hydrophilic surfactant; and/or a polymer selected from the group consisting of polyvinyl alcohol and vinylpyrollidone polymers; and/or a surfactant; and/or an emulsifier in addition to the modified starch.

### Embodiment 90

A personal care composition comprising at least two modified starches selected from the group consisting of:
a) a waxy corn starch that is hydroxypropylated and cross-linked (e.g. by phosphorylation); and
b) a dent corn starch that is hydroxyethylated; and
c) a maltodextrin; and
d) a waxy corn starch that is cross-linked (e.g. by phosphorylation), optionally wherein the composition further comprises propanediol or an ester thereof.

### Embodiment 91

The personal care composition of any of Embodiments 1 or 90, wherein the composition comprises:
a) a waxy corn starch that is hydroxypropylated and cross-linked (e.g. by phosphorylation); and
b) a dent corn starch that is hydroxyethylated; and optionally
c) a maltodextrin.

### Embodiment 92

The personal care composition according to Embodiment 1, wherein the personal care composition is a gel (e.g. an eye gel), the gel comprising:
from about 10-20% by weight bio-1,3-propanediol; and
from about 4 to 8% by weight of a hydroxypropylated and crosslinked (e.g., phosphorylated) waxy corn starch; and
an active agent such as retinyl linoleate in an amount of up to 2% wt; and
optionally one or more of the group consisting of emulsifiers, preservatives, emollients, solvents and thickeners, wherein the weight provided is % wt based on the total weight of the composition.

Embodiment 93 The gel according to Embodiment 92 comprising:
from about 10-20% by weight bio-1,3-propanediol; and
from about 4 to 8% by weight of a hydroxypropylated and crosslinked (e.g., phosphorylated) waxy corn starch; and
from about 1 to 3% emulsifier such as polysorbate; and
from about 1 to 2% preservative; and
up to about 2% wt of an active agent such as retinyl linoleate, wherein the weight provided is % wt based on the total weight of the composition.

### Embodiment 94

The personal care composition according to Embodiment 1 wherein the personal care composition is a serum (e.g. an eye serum), the serum comprising:
from about 10-20% by weight bio-1,3-propanediol; and
from about 3 to 8% by weight of a hydroxypropylated and crosslinked (e.g., phosphorylated) waxy corn starch; and
optionally one or more of the group consisting of emulsifiers, emollients, preservatives, solvents and thickeners.

### Embodiment 95

The serum according to Embodiment 94, comprising:
from about 10-20% by weight bio-1,3-propanediol; and
from about 3 to 8% by weight of a hydroxypropylated and crosslinked (e.g., phosphorylated) waxy corn starch; and
from about 1 to 3% wt emulsifier such as polysorbate; and
from about 1 to 2% preservative; and
up to about 2% wt of an active agent such as retinyl linoleate, wherein the weight provided is % wt based on the total weight of the composition.

### Embodiment 96

The personal care composition according to Embodiment 1 wherein the composition is a lotion (e.g. a face lotion) comprising:
from about 10-20% by weight bio-1,3-propanediol; and
from about 1 to 5% by weight of a modified corn starch selected from one or more of hydroxypropylated and crosslinked (e.g., phosphorylated) waxy corn starch, hydroxyethylated dent corn starch, and maltodextrin; and
optionally one or more of the group consisting of emulsifiers, emollients, preservatives, and solvent, wherein the weight provided is % wt based on the total weight of the composition.

### Embodiment 97

The personal care lotion of Embodiment 96, wherein the lotion is an aqueous lotion, the lotion comprising:
from about 10-20% by weight bio-1,3-propanediol; and
from about 0.5 to 2% by weight of a hydroxypropylated and crosslinked (e.g., phosphorylated) waxy corn starch; and
from about 0.5 to 2% by weight of a hydroxyethylated dent corn starch; and
from about 0.5 to 2% by weight of a maltodextrin; and
optionally one or more of the group consisting of emulsifiers, emollients, preservatives, solvents and thickeners, wherein the weight provided is % wt based on the total weight of the composition.

### Embodiment 98

The aqueous personal care lotion according to Embodiment 97 comprising:
from about 10-20% by weight bio-1,3-propanediol; and
from about 0.5 to 2% by weight of a hydroxypropylated and crosslinked (e.g., phosphorylated) waxy corn starch; and
from about 0.5 to 2% by weight of a hydroxyethylated dent corn starch; and
from about 0.5 to 2% by weight of a maltodextrin;
from about 2 to 8% wt further emulsifier (e.g. glyceryl stearate and one or more of PEG-100 stearate, or sodium stearoyl lactylate); and
from about 12 to 18% wt emollient such as isostearyl isostearate; and
from about 0.5 to 2% wt preservative; and
from about 30-50% wt water, wherein the % provided is % wt based on the total weight of the composition.

### Embodiment 99

The personal care composition according to Embodiment 1, wherein the personal care composition is a cream (e.g. a hand cream), comprising:
from about 10-20% by weight bio-1,3-propanediol; and
from about 5 to 10% by weight of a modified corn starch selected from one or more of hydroxypropylated and crosslinked (e.g., phosphorylated) waxy corn starch, hydroxyethylated dent corn starch and octenyl succinate modified corn starch; and
optionally one or more of the group consisting of emulsifiers, emollients, preservatives, solvents and thickeners, wherein the % provided is % wt based on the total weight of the composition.

### Embodiment 100

The personal care cream according to Embodiment 99, comprising:
from about 10-20% by weight bio-1,3-propanediol; and
from about 2 to 6% by weight of a hydroxypropylated and crosslinked (e.g., phosphorylated) waxy corn starch; and
from about 0.5 to 2% by weight of a hydroxyethylated dent corn starch; and
from about 2 to 4% by weight of an octenyl succinylated corn starch;
optionally one or more of the group consisting of emulsifiers, emollients, preservatives, solvents and thickeners, wherein the % provided is % wt based on the total weight of the composition.

### Embodiment 101

The personal care cream according to Embodiment 100 wherein the cream is an aqueous cream comprising:
from about 10-20% by weight bio-1,3-propanediol; and
from about 2 to 6% by weight of a hydroxypropylated and crosslinked (e.g., phosphorylated) waxy corn starch; and
from about 0.5 to 2% by weight (e.g. about 1% wt) of a hydroxyethylated dent corn starch; and
from about 0.5% to 4% by weight of a sodium starch octenyl succinate;
from about 0.2 to 1% further emulsifier; and
from about 2 to 3% thickener, such as cetyl alcohol; and
from about 12 to 18% emollient such as acetylated hydrogenated vegetable glyceride; and
from about 0.5 to 2% preservative; and
from about 45-65% wt water, wherein the % provided is % wt based on the total weight of the composition.

What is claimed is:

1. A personal care composition comprising:
a propanediol or an ester thereof; and
a modified starch selected from the group consisting of modified corn starch such as modified dent corn starch or modified waxy corn starch; modified wheat starch; modified barley starch; modified tapioca starch; modified triticale starch; modified sorghum starch; modified rice starch; and modified legume starch such as modified pea starch or modified soybean starch.

2. The personal care composition according to claim 1 wherein the propanediol is 1,3-propanediol.

3. The personal care composition according to claim 2 wherein the 1,3-propanediol is biologically derived 1,3-propanediol (i.e. bio-PDO), optionally derived by fermentation methods.

4. The personal care composition according to claim 1 comprising more than one modified starch.

5. The personal care composition according to claim 1 wherein the modified starch is selected from the group consisting of a hydrolysed starch, an oxidized starch, an esterified starch, an etherified starch, an acetylated starch, a C₁₋₁₀alkylated starch, a hydroxyC₁₋₁₀alkylated starch, a crosslinked starch, a phosphorylated starch, a succinylated starch, and a starch that contains two or more of said modifications.

6. The personal care composition according to claim 1 wherein the modified starch is selected from the group consisting of modified dent corn starch, modified waxy corn starch and modified tapioca starch.

7. The personal care composition according to claim 6 wherein the at least one modified starch is hydroxypropylated or hydroxyethylated starch.

8. The personal care composition according to claim 1 wherein the starch is cross-linked, preferably by phosphorylation.

9. The personal care composition according to claim 1 wherein the personal care composition is selected from the group consisting of a gel, a cream, a solution, a spray, a lotion, an ointment, an emulsion, a mousse, a cleanser, a slurry, a serum, or a paste.

10. The personal care composition according to claim 1 wherein the personal care composition is a cosmetic composition, a dermatological care composition, or a body care compositions.

11. The personal care composition according to claim 1 wherein:
the personal care composition is an emulsion;
the propanediol is 1,3-propanediol; and
the modified starch is crosslinked (e.g., phosphorylated) and hydroxypropylated;
optionally wherein the composition does not contain carbomer and/or Xanthan Gum.

12. The personal care composition according to claim 1, wherein:
the personal care composition is a cleanser, optionally a foaming cleanser;
the propanediol is 1,3-propanediol; and
the modified starch is crosslinked (e.g., phosphorylated) and hydroxypropylated.

13. The personal care composition according to claim 1, wherein:
the personal care composition is a gel or serum;
the propanediol is 1,3-propanediol; and
the modified starch is crosslinked (e.g., phosphorylated) and hydroxypropylated.

14. The personal care composition according to claim 1, wherein:
the personal care composition is a cream, preferably an eye cream;
the propanediol is 1,3-propanediol; and
the modified starch is crosslinked (e.g., phosphorylated).

15. The personal care composition according to acclaim 1, wherein:
the personal care composition is an emulsion;
the propanediol is 1,3-propanediol; and
the modified starch is hydroxyethylated.

16. The personal care composition according to claim 1, wherein:
the personal care composition is a sunscreen or makeup;
the propanediol is 1,3-propanediol; and
the modified starch is maltodextrin.

17. The personal care composition according to claim 1 wherein:
the personal care composition is a film forming composition;
the propanediol is 1,3-propanediol; and
the modified starch is maltodextrin.

18. The personal care composition according to claim 1 wherein the composition comprises up to about 50% wt of the modified starch by dry mass relative to the dry mass weight of the personal care composition, optionally from about 1-30%, such as from about 4 to 7% wt or from 10-30% wt by dry mass, e.g. from about 12-28% wt by dry mass.

19. The personal care composition according to claim 1 wherein the composition comprises up to about 80% wt of propanediol or ester thereof by dry mass relative to the dry mass weight of the personal care composition, optionally from about 10-65%, such as from about 20-65% wt by dry mass.

20. A personal care composition comprising at least two modified starches selected from the group consisting of:
a) a waxy corn starch that is hydroxypropylated and cross-linked (e.g. by phosphorylation); and
b) a dent corn starch that is hydroxyethylated; and
c) a maltodextrin; and
d) a waxy corn starch that is cross-linked (e.g. by phosphorylation),
optionally wherein the composition further comprises propanediol or an ester thereof.
